SEQUENCE LISTING (7 sequences)

SEQ ID NO: 1 - protein, 12 aa, Artificial Sequence
  Artificially synthesized peptide
HIITDPNMAEYL

SEQ ID NO: 2 - protein, 12 aa, Artificial Sequence
  Artificially synthesized peptide
SYTQRADSTTLH

SEQ ID NO: 3 - protein, 12 aa, Artificial Sequence
  Artificially synthesized peptide
GYGFSNTNSFFV

SEQ ID NO: 4 - protein, 12 aa, Artificial Sequence
  Artificially synthesized peptide
SHMQNRPASDEH

SEQ ID NO: 5 - protein, 12 aa, Artificial Sequence
  Artificially synthesized peptide
AYNAGSILENNF

SEQ ID NO: 6 - protein, 12 aa, Artificial Sequence
  Artificially synthesized peptide
LVPDRMTAISRA

SEQ ID NO: 7 - protein, 12 aa, Artificial Sequence
  Artificially synthesized peptide
NSLRNYDFLITM

(19) United States
(12) Patent Application Publication
(10) Pub. No.: US 2017/0258930 A1
(43) Pub. Date: Sep. 14, 2017

(54) SKIN PENETRATING PEPTIDES (SPPS) AND METHODS OF USE THEREFOR

(71) Applicants: The Regents of the University of California, Oakland, CA (US); CTX Technology Inc., Tucson, AZ (US)

(72) Inventors: John A. Muraski, Tucson, AZ (US); Samir Mitragotri, Santa Barbara, CA (US); Sunny Kumar, Santa Barbara, CA (US)

(73) Assignees: The Regents of the University of California, Oakland, CA (US); CTX Technology Inc., Tucson, AZ (US)

(21) Appl. No.: 15/506,560

(22) PCT Filed: Aug. 27, 2015
(86) PCT No.: PCT/US2015/047160

§ 371 Date: Feb. 24, 2017

Related U.S. Application Data
(60) Provisional application No. 62/042,546, filed on Aug. 27, 2014.

(51) Int. Cl. A61K 48/00; C07K 4/00; C07K 7/04; A61K 38/04; A61K 49/00; A61K 47/48; A61K 38/16

(52) U.S. Cl. CPC A61K 47/48246; A61K 38/04; C07K 4/00; C07K 7/04

(57) ABSTRACT

The present disclosure provides peptides and peptide compositions, optionally in the from of a vaccine, which facilitate the delivery of an active agent or an active agent carrier wherein the compositions are capable of penetrating the stratum corneum (SC) and/or the cellular membranes of viable cells. Also provided are methods of employing the peptides and peptide compositions to deliver active agents; treat diseases or disorders; and inducing immune responses.

### CROSS REFERENCE TO RELATED APPLICATION

This application is based on and claims priority to U.S. Provisional Patent Application Ser. No. 62/042,546, filed Aug. 27, 2014, the disclosure of which is incorporated by reference herein in its entirety.

### TECHNICAL FIELD

The presently disclosed subject matter relates to peptides, optionally peptides conjugated to one or more active agents and/or active agent carriers comprising the active agent(s). Also provided are compositions comprising the presently disclosed peptides and/or conjugates, wherein the compositions are capable of penetrating a stratum corneum (SC) layer when contacted therewith or penetrating a cell when contacted therewith, as well as methods for employing the claimed peptides, conjugates, and/or compositions to deliver active agents to subjects.

### BACKGROUND

Skin is the largest organ of the human body. It is also host to numerous dermatological diseases, which collectively represent a large category of human health conditions. Accordingly, successful delivery of therapeutics and other active agents including but not limited to small molecules, macromolecules, vaccines, polynucleotides (e.g., siRNAs, antisense oligonucleotides, etc.), cosmeceuticals, etc., into skin has become a topic of active research and development.

Topical delivery of these molecules is extremely challenging, however, and with some exceptions, has been very difficult to accomplish. The primary challenge is poor skin penetration of macromolecules. Among various physico-chemical methods proposed to enhance penetration of macromolecules, peptide carriers have emerged as potential candidates owing to their simplicity of use, diversity, and potential ability to target cellular subtypes within the skin. Several peptides including TAT, polyarginine, meganin, and penetratin, which were initially identified for delivering drugs into the cytoplasm of cells, have been tested for penetration across the stratum corneum (SC), and a few have shown some efficacy in delivering small molecules into the epidermis.

In contrast, very few peptides have been specifically shown to penetrate the SC and possess the ability to enhance systemic uptake of topically applied drugs. PCT International Patent Application Publication No. WO 2007/035474 describes the TD series of peptides designed to enhance transdermal delivery of pharmaceutically active agents, and U.S. Pat. No. 8,518,871 to Hsu et al. describe Skin Permeating and Cell Entering (SPACE) Peptides, which facilitate the delivery of active agents and active agent carriers across the SC and/or the cellular membranes of viable cells. Although several peptides are known to penetrate cellular membranes and a few to penetrate the SC, peptides that simultaneously enhance the penetration of macromolecules and other actives across the SC and/or across the cellular membranes of viable epidermal and dermal cells are needed.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides peptides and peptide compositions that facilitate the delivery of active agents and/or active agent carriers into and/or across the SC and/or the cellular membranes of viable cells.

In some embodiments, a composition of the presently disclosed subject matter comprises a peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7). In some embodiments, the peptide is associated with and/or conjugated to an active agent or an active agent carrier comprising the active agent. In some embodiments, the composition is capable of penetrating a stratum corneum (SC) layer when contacted therewith or penetrating a cell when contacted therewith. In some embodiments, the composition is capable of penetrating the cellular membrane of a cell selected from the group consisting of a viable non-human animal cell, a viable human cell, a viable epidermal cell, a viable dermal cell, and a viable immunological cell.

In some embodiments of the presently disclosed subject matter, the active agent comprises a protein, a nucleic acid, a pharmaceutical compound, a detectable agent, a nanoparticle, or a low molecular weight compound. In some embodiments, the protein comprises an antibody or a fragment thereof comprising at least one paratope. In some embodiments, the nucleic acid comprises DNA, RNA, or a combination thereof. In some embodiments, the RNA is an interfering RNA including but not limited to an shRNA, an miRNA, and/or an siRNA. In some embodiments, the siRNA is directed against a gene product selected from the group consisting of an IL-10 gene product, a CD86 gene product, a KRT6a gene product, a TNFR1 gene product, and a TACE gene product. In some embodiments, the siRNA is a mutation-specific siRNA.

In some embodiments of the presently disclosed subject matter, the active agent comprises a pharmaceutical compound and/or a detectable agent. In some embodiments, the detectable agent comprises a fluorescent label and/or a radioactive label.

In some embodiments of the presently disclosed subject matter, the active agent is a nanoparticle.

In some embodiments of the presently disclosed subject matter, the active agent is a low molecular weight compound.

In some embodiments of the presently disclosed subject matter, the peptide is conjugated to the active agent and/or is conjugated to an active agent carrier comprising the active agent. In some embodiments, the peptide is associated with the active agent and/or the active agent carrier comprising the active agent via hydrophobic, electrostatic, and/or van der Walls interactions.

In some embodiments of the presently disclosed subject matter, the active agent carrier is selected from the group consisting of a liposome, a nanoparticle, and a polymeric micelle.

In some embodiments of the presently disclosed subject matter, the peptide is from 9 to 11 amino acids in length. In some embodiments, the peptide is from about 12-15 amino acids in length. In some embodiments, the peptide is from about 16-19 amino acids in length.

In some embodiments, the presently disclosed subject matter provides an isolated peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7). In some embodiments, the peptide comprises repeat units of one or more of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7). In some embodiments, the unit is repeated 2 to 50 times. In some embodiments, each unit is separated by an intervening peptide sequence. In some embodiments, the isolated peptide is from 9 to 11 amino acids in length. In some embodiments, the isolated peptide from about 12-15 amino acids in length. In some embodiments, the isolated peptide is from about 16-19 amino acids in length.

The presently disclosed subject matter also provides methods for delivering active agents to subjects. In some embodiments, the methods comprise administering to the subject a composition comprising a peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7), wherein the peptide is conjugated to and/or associated with an active agent or an active agent carrier comprising the active agent. In some embodiments, the administration is topical administration. In some embodiments, the composition is capable of penetrating the stratum corneum (SC) of the subject and/or penetrating a cell of the subject. In some embodiments, the composition is capable of penetrating the SC of the subject and penetrating the cell of the subject. In some embodiments, the composition is capable of penetrating the cellular membrane of a cell selected from the group consisting of a viable non-human animal cell, a viable human cell, a viable epidermal cell, a viable dermal cell, and a viable immunological cell.

In some embodiments of the presently disclosed methods, the active agent comprises a protein, a nucleic acid, a pharmaceutical compound, a detectable agent, a nanoparticle, or a low molecular weight compound. In some embodiments, the protein comprises an antibody or a fragment thereof comprising at least one paratope. In some embodiments, the nucleic acid is DNA, RNA, or a combination thereof. In some embodiments, the RNA is an interfering RNA, including but not limited to shRNA, miRNA, siRNA, or any combination thereof. In some embodiments, the nucleic acid comprises an siRNA, and the siRNA is directed against a gene product selected from the group consisting of an IL-10 gene product, a CD86 gene product, a KRT6a gene product, a TNFR1 gene product, and a TACE gene product. In some embodiments, the siRNA is a mutation-specific siRNA.

In some embodiments of the presently disclosed methods, the active agent comprises a pharmaceutical compound.

In some embodiments of the presently disclosed methods, the active agent comprises a detectable agent. In some embodiments, the detectable agent comprises a fluorescent label and/or a radioactive label.

In some embodiments of the presently disclosed methods, the active agent is a nanoparticle.

In some embodiments of the presently disclosed methods, the active agent is a low molecular weight compound.

In some embodiments of the presently disclosed methods, the peptide is conjugated to the active agent. In some embodiments, the peptide is conjugated to an active agent carrier comprising the active agent. In some embodiments, the active agent carrier is selected from the group consisting of a liposome, a nanoparticle, and a polymeric micelle.

In some embodiments of the presently disclosed methods, the peptide is associated with the active agent and/or the active agent carrier comprising the active agent via hydrophobic, electrostatic, and/or van der Walls interactions.

In some embodiments, the presently disclosed subject matter provides methods for treating a subject having a dermatological disease. In some embodiments, the presently disclosed methods comprise administering to the subject a composition comprising a peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7). In some embodiments, the peptide is conjugated to and/or associated with a dermatological active agent and/or a dermatological active agent carrier comprising the active agent. In some embodiments, the composition is capable of penetrating the stratum corneum (SC) of the subject, penetrating a cell of the subject, or both. In some embodiments, the administration is topical administration.

In some embodiments of the presently disclosed methods, the composition is capable of penetrating the cellular membrane of a cell selected from the group consisting of a viable non-human animal cell, a viable human cell, a viable epidermal cell, a viable dermal cell, and a viable immunological cell.

In some embodiments of the presently disclosed methods, the active agent comprises a protein, a nucleic acid, a pharmaceutical compound, a detectable agent, a nanoparticle, or a low molecular weight compound. In some embodiments, the protein comprises an antibody or a fragment thereof comprising at least one paratope. In some embodiments, the nucleic acid is DNA, RNA, or any combination thereof. In some embodiments, the nucleic acid is RNA, optionally an interfering RNA. In some embodiments, the interfering RNA is selected from the group consisting of shRNA, miRNA, siRNA, and any combination thereof. In some embodiments, the siRNA is directed against a gene product selected from the group consisting of an IL-10 gene product, a CD86 gene product, a KRT6a gene product, a TNFR1 gene product, and a TACE gene product. In some embodiments, the siRNA is a mutation-specific siRNA.

In some embodiments of the presently disclosed methods, the active agent comprises a pharmaceutical compound and/or a detectable agent. In some embodiments, the detectable agent comprises a fluorescent label and/or a radioactive label.

In some embodiments of the presently disclosed methods, the active agent is a nanoparticle or a low molecular weight compound.

In some embodiments, the peptide is conjugated to the active agent, is conjugated to an active agent carrier comprising the active agent, or both. In some embodiments, the active agent carrier is selected from the group consisting of a liposome, a nanoparticle, and a polymeric micelle.

In some embodiments of the presently disclosed methods, the peptide is associated with the active agent and/or the active agent carrier comprising the active agent via hydrophobic, electrostatic, and/or van der Walls interactions.

The presently disclosed subject matter also provides in some embodiments methods for treating a subject having, suspected of having, or susceptible to a disorder resulting at least in part from expression of an mRNA. In some embodiments, the presently disclosed methods comprise administering to the subject a composition comprising a peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7). In some embodiments, the peptide is conjugated to and/or associated with an interfering RNA that targets the mRNA and/or a carrier comprising the interfering RNA; the composition is capable of penetrating the stratum corneum (SC) of the subject and/or a cell of the subject, and the administering step results in expression of the mRNA being attenuated thereby. In some embodiments, the composition is capable of penetrating both the SC of the subject and the cell of the subject. In some embodiments, the administration is topical administration.

In some embodiments, the presently disclosed subject matter also provides methods for treating a subject having, suspected of having, or susceptible to developing a disorder resulting at least in part from expression of an mRNA, comprising administering to the subject a composition comprising a peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7). In some embodiments the peptide is associated with an interfering RNA that targets the mRNA or a carrier comprising the interfering RNA; the association results from hydrophobic, electrostatic, and/or van der Walls interactions; the composition is capable of penetrating the stratum corneum (SC) of the subject and/or a cell of the subject; and the administering step results in expression of the mRNA being attenuated thereby. In some embodiments, the composition is capable of penetrating both the SC of the subject and the cell of the subject. In some embodiments, the administration is topical administration.

In some embodiments, the presently disclosed subject matter also provides methods for attenuating expression of an mRNA of a subject in need thereof. In some embodiments, the presently disclosed methods comprise administering to the subject a composition comprising a peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7). In some embodiments, the peptide is conjugated to an siRNA targeted to the mRNA and/or a carrier comprising the siRNA targeted to the mRNA; the composition is capable of penetrating the stratum corneum (SC) of the subject, a cell of the subject, or both; and the administering step results in expression of the mRNA being attenuated thereby. In some embodiments, the mRNA is an IL-10 mRNA and the siRNA is an IL-10 siRNA, the mRNA is a CD86 mRNA and the siRNA is a CD86 siRNA, the mRNA is a KRT6a mRNA and the siRNA is KRT6a siRNA, the mRNA is a TNFR 1 mRNA and the siRNA is a TNFR1 siRNA, and/or the mRNA is a TACE mRNA and the siRNA is a TACE siRNA. In some embodiments, the administration is topical administration.

In some embodiments of the presently disclosed methods, the peptide is associated with an siRNA targeted to the mRNA and/or a carrier comprising the siRNA targeted to the mRNA; the association results from hydrophobic, electrostatic, and/or van der Walls interactions; the composition is capable of penetrating the stratum corneum (SC) of the subject and/or a cell of the subject; and the administering step results in expression of the mRNA being attenuated thereby. In some embodiments, the mRNA is an IL-10 mRNA and the siRNA is an IL-10 siRNA, the mRNA is a CD86 mRNA and the siRNA is a CD86 siRNA, the mRNA is a KRT6a mRNA and the siRNA is KRT6a siRNA, the mRNA is a TNFR 1 mRNA and the siRNA is a TNFR1 siRNA, and/or the mRNA is a TACE mRNA and the siRNA is a TACE siRNA. In some embodiments, the composition is capable of penetrating both the SC and the cell of the subject. In some embodiments, the administration is topical administration.

In some embodiments, the presently disclosed subject matter provides compositions comprising a peptide consisting essentially of or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7). In some embodiments, the peptide is associated with and/or conjugated to an active agent and/or an active agent carrier comprising the active agent. In some embodiments, the composition is capable of penetrating a stratum corneum (SC) layer when contacted therewith or penetrating a cell when contacted therewith.

Thus, in some embodiments it is an object of the presently disclosed subject matter to provide compositions and methods for delivering active agents to subjects.

An object of the presently disclosed subject matter having been stated herein above, and which is achieved in whole or in part by the presently disclosed subject matter, other objects will become evident as the description proceeds when taken in connection with the accompanying drawings as best described herein below.

### DETAILED DESCRIPTION

The present subject matter will be now be described more fully hereinafter with reference to the accompanying Examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### I. General Considerations

Skin, the largest organ in the human body is a potential site for local and systemic delivery of therapeutics. However, the skin's outermost layer, the stratum corneum (SC), forms a stringent barrier for transport of hydrophilic and macromolecules into and across the skin. As a result only a few small (<500 Da), lipophilic (Log P=1-3) molecules can be passively delivered through the skin. In particular, it is difficult to deliver large drug molecules such as proteins and nucleotide-based (e.g., siRNA) medicines due to their large size and hydrophilic nature.

To address these limitations, several physico-chemical skin penetration enhancement techniques have been proposed. However, the majority of these techniques are toxic at higher concentrations (e.g., chemical enhancers), invasive in nature (microneedles, tape stripping, etc.), inconvenient and/or costly for subjects, and/or difficult to develop into a delivery system (e.g., ultrasound, iontophoresis, etc.). In contrast, peptides acting as skin penetration enhancers have recently emerged as a simple, noninvasive strategy for macromolecules delivery into and across the skin. Several peptides which are known to penetrate cellular membranes for drug delivery, including TAT, polyarginine, meganin, and penetratin, have been tested for small molecule delivery into the skin (up to epidermis). On the other hand, phage display peptide library (PDL) screening on mouse and porcine skin have identified peptides (e.g., TD-1, SPACE and T2 peptides) that could cross human, porcine, and murine skin.

Interestingly, the SPACE peptides disclosed in U.S. Pat. No. 8,518,871 (incorporated herein by reference in its entirety) has shown penetrating the skin as well as the cell membranes of various cell types. In addition, SPACE peptides were found to be capable of delivering macromolecules, such as but not limited to siRNA, into skin and cells for therapeutic purposes.

Disclosed herein are additional phage display peptide library (PDL) screening experiments with porcine skin using a random linear 12 amino acid peptide library (PDL). Several 12-mer peptides were identified that could transport M13 bacteriophages (1000 nm long and 10 nm wide) across porcine skin. One such peptide had the amino acid sequence HIITDPNMAEYL (SEQ ID NO: 1), and is referred to herein as a Skin Penetrating Peptide (SPP).

### II. Definitions

Before the presently disclosed subject matter is further described, it is to be understood that the presently disclosed subject matter is not limited to particular embodiments described, as such can, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which the presently disclosed subject matter belongs. Although any methods and materials similar or equivalent to those described herein can also be used in the practice or testing of the presently disclosed subject matter, exemplary methods and materials are now described.

All references listed in the instant disclosure, including but not limited to all patents, patent applications and publications thereof, scientific journal articles, and database entries (including but not limited to GENBANK® biosequence database entries and all annotations and cited references presented therein) are incorporated herein by reference in their entireties to the extent not inconsistent herewith and to the extent that they supplement, explain, provide a background for, or teach methodology, techniques, and/or compositions employed herein. To the extent any of the references disclosed and incorporated herein conflict with the instant disclosure, the instant disclosure controls.

The publications discussed herein are provided solely for their disclosure prior to the filing date of the present application. Nothing herein is to be construed as an admission that the presently disclosed subject matter is not entitled to antedate such publication by virtue of prior invention. Further, the dates of publication provided can be different from the actual publication dates, which might need to be independently confirmed.

It must be noted that as used herein and in the appended claims, the singular forms “a”, “and”, and “the” include plural referents unless the context clearly dictates otherwise. Thus, for example, reference to “a peptide” includes a plurality of such peptides and reference to the “agent” includes reference to one or more agents and equivalents thereof known to those skilled in the art, and so forth.

It will be appreciated that throughout this present disclosure reference is made to amino acids according to the single letter or three letter code. For convenience, the single and three letter codes for each amino acid, as well as functionally equivalent codons therefor, are provided in Table 1 below:

TABLE 1
Amino Acid Abbreviations, Codes, and Functionally
Equivalent Codons

| | 3- | 1- | |
|---|---|---|---|
| Amino Acid | Letter | Letter | Codons |
| Alanine | Ala | A | GCA GCC GCG GCU |
| Arginine | Arg | R | AGA AGG CGA CGC CGG |
| | | | CGU |
| Asparagine | Asn | N | AAC AAU |
| Aspartic Acid | Asp | D | GAC GAU |
| Cysteine | Cys | C | UGC UGU |
| Glutamic acid | Glu | E | GAA GAG |
| Glutamine | Gln | Q | CAA CAG |
| Glycine | Gly | G | GGA GGC GGG GGU |
| Histidine | His | H | CAC CAU |
| Isoleucine | Ile | I | AUA AUC AUU |
| Leucine | Leu | L | UUA UUG CUA CUC CUG |
| | | | CUU |
| Lysine | Lys | K | AAA AAG |
| Methionine | Met | M | AUG |
| Phenylalanine | Phe | F | UUC UUU |
| Proline | Pro | P | CCA CCC CCG CCU |
| Serine | Ser | S | ACG AGU UCA UCC UCG |
| | | | UCU |
| Threonine | Thr | T | ACA ACC ACG ACU |
| Tryptophan | Trp | W | UGG |
| Tyrosine | Tyr | Y | UAC UAU |
| Valine | Val | V | GUA GUC GUG GUU |

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

As used herein, the phrase “active agent” refers to an agent, including but not limited to a protein, peptide, nucleic acid (such as but not limited to nucleotides, nucleosides, and analogues thereof) or small molecule drug, which provides a desired pharmacological effect upon administration to a subject, e.g., a human or a non-human animal, either alone or in combination with other active or inert components. Included in the above definition are precursors, derivatives, analogues, and prodrugs of active agents. The phrase “active agent” can in some embodiments also be used herein to refer generally to any agent, e.g., a protein, peptide, nucleic acid (including but not limited to nucleotides, nucleosides, and analogues thereof) or small molecule drug, conjugated or associated with a penetrating peptide as described herein and/or attached to or encompassed by an active agent carrier as described herein.

The term “conjugated” as used in the context of the penetrating peptide compositions described herein refers to a covalent and/or ionic interaction between two entities, e.g., molecules, compounds, or combinations thereof.

The term “associated” as used in the context of the penetrating peptide compositions described herein refers to a non-covalent interaction between two entities, e.g., molecules, compounds, or combinations thereof, mediated by one or more of hydrophobic, electrostatic, and/or van der Walls interactions.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, such as but not limited to fusion proteins with a heterologous amino acid sequence; fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “antibody” and “immunoglobulin” include antibodies or immunoglobulins of any isotype, fragments of antibodies that retain specific binding to antigen, including but not limited to Fab, Fv, scFv, and Fd fragments, chimeric antibodies, humanized antibodies, single-chain antibodies, and fusion proteins including an antigen-binding portion of an antibody and a non-antibody protein. The antibodies can in some embodiments be detectably labeled, e.g., with a radioisotope, an enzyme which generates a detectable product, a fluorescent protein, and the like. The antibodies can in some embodiments be further conjugated to other moieties, such as members of specific binding pairs, e.g., biotin (member of biotin-avidin specific binding pair), and the like. Also encompassed by the terms are Fab′, Fv, F(ab′)₂, and other antibody fragments that retain specific binding to antigen.

Antibodies can exist in a variety of other forms including, for example, Fv, Fab, and (Fab′)₂, as well as bi-functional (i.e. bi-specific) hybrid antibodies (see e.g., Lanzavecchia et al., Eur. J. Immunol. 17, 105 (1987)) and in single chains (see e.g., Huston et al., Proc. Natl. Acad. Sci. U.S.A., 85, 5879-5883 (1988) and Bird et al., Science, 242, 423-426 (1988), each of which is incorporated herein by reference in its entirety). See generally, Hood et al., Immunology, Benjamin, N.Y., 2nd ed. (1984), and Hunkapiller and Hood, Nature, 323, 15-16 (1986).

The phrases “nucleic acid”, “nucleic acid molecule”, and “polynucleotide” are used interchangeably and refer to a polymeric form of nucleotides of any length, deoxyribonucleotides and/or ribonucleotides, and/or analogs thereof. The terms encompass, e.g., DNA, RNA, and modified forms thereof. Polynucleotides can in some embodiments have any three-dimensional structure, and can perform any function, known and/or unknown. Non-limiting examples of polynucleotides include a gene, a gene fragment, exons, introns, messenger RNA (mRNA), transfer RNA (tRNA), ribosomal RNA (rRNA), ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule can be linear or circular.

“RNA interference” (RNAi) is a process by which double-stranded RNA (dsRNA) is used to silence gene expression. Without intending to be bound by any particular theory, RNAi begins with the cleavage of longer dsRNAs into small interfering RNAs (siRNAs) by dicer, an RNaseIII-like enzyme. siRNAs are dsRNAs that are generally in some embodiments about 19 to 28 nucleotides, or in some embodiments about 20 to 25 nucleotides, or in some embodiments about 21 to 23 nucleotides in length, and often contain 2-3 nucleotide 3′ overhangs and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. The siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA can in some embodiments be designed (e.g., via decreased siRNA duplex stability at the 5′ end of the antisense strand) to favor incorporation of the antisense strand into RISC.

RISC-mediated cleavage of mRNAs having a sequence at least partially complementary to the guide strand leads to a decrease in the steady state level of that mRNA and of the corresponding protein encoded by the mRNA. Alternatively, RISC can also decrease expression of the corresponding protein via translational repression without cleavage of the target mRNA. Other RNA molecules can interact with RISC and silence gene expression. Examples of other RNA molecules that can interact with RISC include short hairpin RNAs (shRNAs), single-stranded siRNAs, microRNAs (miRNAs), and dicer-substrate 27-mer duplexes, RNA molecules containing one or more chemically modified nucleotides, one or more deoxyribonucleotides, and/or one or more non-phosphodiester linkages. The term “siRNA” as used herein refers to a double-stranded interfering RNA unless otherwise noted. For purposes of the present disclosure, all RNA molecules that can interact with RISC and participate in RISC-mediated changes in gene expression will be referred to as “interfering RNAs.” siRNAs, shRNAs, miRNAs, and dicer-substrate 27-mer duplexes are, therefore, subsets of “interfering RNAs”.

A “substitution” results from the replacement of one or more amino acids or nucleotides by different amino acids or nucleotides, respectively as compared to an amino acid sequence or nucleotide sequence of a polypeptide. If a substitution is conservative, the amino acid that is substituted into a polypeptide has similar structural or chemical properties (e.g., charge, polarity, hydrophobicity, and the like) to the amino acid that it is substituting. Conservative substitutions of naturally occurring amino acids usually result in a substitution of a first amino acid with second amino acid from the same group as the first amino acid, where exemplary amino acid groups are as follows: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral non-polar amino acids such as alanine, leucine, isoleucine, valine, proline, phenylalanine, tryptophan, and methionine. In some embodiments, polypeptide variants can have “non-conservative” changes, where the substituted amino acid differs in structural and/or chemical properties.

A “deletion” is defined as a change in either amino acid or nucleotide sequence in which one or more amino acid or nucleotide residues, respectively, are absent as compared to an amino acid sequence or nucleotide sequence of a naturally occurring polypeptide. In the context of a polypeptide or polynucleotide sequence, a deletion can involve deletion of in some embodiments 2, in some embodiments 5, in some embodiments 10, in some embodiments up to 20, in some embodiments up to 30, or in some embodiments up to 50 or more amino acids, taking into account the length of the polypeptide or polynucleotide sequence being modified.

An “insertion” or “addition” is that change in an amino acid or nucleotide sequence which has resulted in the addition of one or more amino acid or nucleotide residues, respectively, as compared to an amino acid sequence or nucleotide sequence of a naturally occurring polypeptide. “Insertion” generally refers to addition to one or more amino acid residues within an amino acid sequence of a polypeptide, while “addition” can be an insertion or refer to amino acid residues added at the N- or C-termini. In the context of a polypeptide or polynucleotide sequence, an insertion or addition can be of in some embodiments up to 10, in some embodiments up to 20, in some embodiments up to 30, or in some embodiments up to 50 or more amino acids.

“Non-native”, “non-endogenous”, and “heterologous”, in the context of a polypeptide, are used interchangeably herein to refer to a polypeptide having an amino acid sequence or, in the context of an expression system or a viral particle, present in an environment different to that found in nature.

“Exogenous” in the context of a nucleic acid or polypeptide is used to refer to a nucleic acid or polypeptide that has been introduced into a host cell. “Exogenous” nucleic acids and polypeptides can be native or non-native to the host cell, where an exogenous, native nucleic acid, or polypeptide provides for elevated levels of the encoded gene product or polypeptide in the recombinant host cell relative to that found in the host cell prior to introduction of the exogenous molecule.

As used herein, the terms “determining”, “measuring”, “assessing”, and “assaying” are used interchangeably and include both quantitative and qualitative determinations.

As used herein the term “isolated”, when used in the context of an isolated compound (including but not limited to nucleic acids, polypeptides, peptides, etc.) refers to a compound of interest that is in an environment different from that in which the compound naturally occurs. “Isolated” is meant to include compounds that are within samples that are substantially enriched for the compound of interest and/or in which the compound of interest is partially or substantially purified.

As used herein, the phrase “substantially pure” refers to a compound that is removed from its natural environment and is in some embodiments at least 60% free, in some embodiments at least 75% free, in some embodiments at least 80% free, in some embodiments at least 85% free, in some embodiments at least 90% free, in some embodiments at least 95% free, in some embodiments at least 97% free, and in some embodiments at least 99% free from other components with which it is naturally associated.

As used herein, the phrase “coding sequence” and a sequence that “encodes” a selected peptide or polypeptide is a nucleic acid molecule which is transcribed (in the case of DNA) and translated (in the case of mRNA) into a peptide or polypeptide, for example, in vivo when placed under the control of appropriate regulatory sequences (or “control elements”). The boundaries of the coding sequence are typically determined by a start codon at or near the 5′ end of the nucleic acid and a translation stop codon at or near the 3′ end of the nucleic acid. A coding sequence can include, but is not limited to, cDNA from viral, prokaryotic, or eukaryotic mRNA, genomic DNA sequences from viral, prokaryotic, or eukaryotic DNA, and synthetic DNA sequences. A transcription termination sequence can be located downstream of (i.e., 3′ to) the coding sequence. Other “control elements” can also be associated with a coding sequence. A DNA sequence encoding a peptide or polypeptide can in some embodiments be optimized for expression in a selected host cell by using the optimized codons for the selected host cell to represent the DNA copy of the desired peptide polypeptide coding sequence. Optimized codons for various species are known (see e.g., the GenScript Codon Usage Frequency Table Tool available from the website of GenScript USA Inc. of Piscataway, N.J., United States of America.

As used herein, the phrase “encoded by” refers to a nucleic acid sequence that codes for a gene product, such as a peptide, polypeptide, or other nucleic acid (e.g., an siRNA). Where the gene product is a polypeptide, the polypeptide sequence or a portion thereof contains an amino acid sequence of in some embodiments at least 3 to 5 amino acids, in some embodiments at elast 8 to 10 amino acids, and in some embodiments at least 15 to 20 amino acids from a polypeptide encoded by the nucleic acid sequence.

As used herein, the phases “operably linked” and “operatively linked” refer to a functional linkage between a nucleic acid expression control sequence (such as but not limited to a promoter, or array of transcription factor binding sites) and a second nucleic acid sequence, wherein the expression control sequence by its presence influences transcription and/or translation of the nucleic acid corresponding to the second sequence. An operably linked promoter or other control element need not be contiguous with the coding sequence, so long as it functions to influence the expression thereof. For example, intervening untranslated yet transcribed sequences can be present between a promoter sequence and a coding sequence, and the promoter sequence is still considered “operably linked” to the coding sequence.

As used herein, the phrase “nucleic acid construct” refers to a nucleic acid sequence that has been generated to comprise one or more functional units at least two of which are not found together in nature. Examples include circular, linear, double-stranded, extrachromosomal DNA molecules (plasmids), cosmids (plasmids containing COS sequences from lambda phage), viral genomes including non-native nucleic acid sequences, and the like.

As used herein, the term “vector” refers to a nucleic acid molecule that is capable of transferring nucleic acid sequences to target cells and/or host cells. Typically, the phrases “vector construct”, “expression vector”, and “gene transfer vector” refer to any nucleic acid molecule (in some embodiments, a recombinantly produced nucleic acid molecule) that is capable of directing the expression of a nucleic acid sequence of interest and that can transfer a nucleic acid sequence to a target and/or host cell, which in some embodiments can be accomplished by genomic integration of all or a portion of the vector, or in some embodiments transient or inheritable maintenance of the vector as an extrachromosomal element. Thus, the phrases includes cloning vectors, expression vehicles, integrating vectors, and the like.

An “expression cassette” includes any nucleic acid construct capable of directing the expression of a nucleic acid sequence of interest (including but not limited to a coding sequence of interest, an interfering RNA sequence of interest, etc.) that is operably linked to a promoter of the expression cassette. Such expression cassettes can be constructed into a “vector”, “vector construct”, “expression vector”, and/or “gene transfer vector” in order to transfer the expression cassette into a target and/or host cell. Thus, the phrase includes, but is not limited to, cloning and expression vehicles, viral vectors, etc.

The terms “identical” and “percent identity” in the context of two or more nucleotide or peptide/polypeptide sequences, refer to two or more sequences or subsequences that are the same or have a specified percentage of nucleotides or amino acids that are the same, when compared and aligned for maximum correspondence, as measured using one of the sequence comparison algorithms disclosed herein or by visual inspection.

The term “substantially identical”, in the context of two nucleotide or peptide/polypeptide sequences, refers to two or more sequences or subsequences that have in some embodiments at least 60%, in some embodiments at least 65%, in some embodiments at least 70%, in some embodiments at least 75%, in some embodiments at least 80%, in some embodiments at least 85%, in some embodiments at least 90%, in some embodiments at least 95%, in some embodiments at least 97%, and in some embodiments at least 99% nucleotide or amino acid identity, when compared and aligned for maximum correspondence, as measured using one of the sequence comparison algorithms described herein below or by visual inspection. In some embodiments, the substantial identity exists in nucleotide or amino acid sequences of at least 5, 10, 15, 20, 25, 30, 35, 40, or 45 nucleotides or amino acids. Alternatively, the substantial identity can exists in nucleotide or amino acid sequences of in some embodiments at least 50 nucleotides or amino acids, in some embodiments at least about 100 nucleotides or amino acids, in some embodiments at least about 150 nucleotides or amino acids, and in some embodiments in nucleotide or amino acids sequences comprising full length sequences (e.g., full length coding sequences and/or the full length amino acid sequences encoded thereby) of a reference nucleotide or peptide/polypeptide sequence.

For sequence comparisons, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981) Adv Appl Math 2:482-489, by the homology alignment algorithm of Needleman & Wunsch (1970) J Mol Biol 48:443-453, by the search for similarity method of Pearson & Lipman (1988) Proc Natl Acad Sci USA 85:2444-2448, by computerized implementations of these algorithms (GAP, BESTFIT, FASTA, and TFASTA, in the PIPELINE® Pilot Sequence Analysis Component Collection available from Accelrys Inc., San Diego, Calif., United States of America), or by visual inspection. See generally, Ausubel (1995) Short Protocols in Molecular Biology, 3rd ed. Wiley, New York, N.Y., United States of America.

In some embodiments, an algorithm for determining percent sequence identity and sequence similarity is the BLAST algorithm, which is described by Altschul et al. (1990) J Mol Biol 215:403-410. Software for performing BLAST analyses is publicly available through the website of the National Center for Biotechnology Information (NCBI). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff (1992) Proc Natl Acad Sci USA 89:10915-10919.

In addition to calculating percent sequence identity, the BLAST algorithm also performs a statistical analysis of the similarity between two sequences. See e.g., Karlin & Altschul (1993) Proc Natl Acad Sci USA 90:5873-5877. One measure of similarity provided by the BLAST algorithm is the smallest sum probability (P(N)), which provides an indication of the probability by which a match between two nucleotide or amino acid sequences would occur by chance. By way of example and not limitation, a test nucleic acid or amino acid sequence can considered similar to a reference sequence if the smallest sum probability in a comparison of the test nucleic acid or amino acid sequence to the reference nucleic acid or amino acid sequence is in some embodiments less than about 0.1, in some embodiments less than about 0.01, and in some embodiments less than about 0.001.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

In some embodiments, an exemplary nucleotide sequence employed in the methods disclosed herein comprises sequences that are complementary to a target sequence (e.g., are the reverse-complement of a target sequence), the complementary regions being capable of forming a duplex of in some embodiments at least about 10 to 50 basepairs. By way of example and not limitation, one strand can comprise a nucleic acid sequence of at least 15, 16, 17, 18, 19, or 20 contiguous bases having a nucleic acid sequence of a nucleic acid molecule of the presently disclosed subject matter. In some embodiments, one strand can comprise a nucleic acid sequence comprising 10, 12, 15 to 18 nucleotides, or even longer where desired, such as 19, 20, 21, 22, 25, or 30 nucleotides or up to the full length of any of target sequence. Such fragments can be readily prepared by, for example, directly synthesizing the fragment by chemical synthesis, by application of nucleic acid amplification technology, or by introducing selected sequences into recombinant vectors for recombinant production.

In some embodiments, an inhibitory nucleic acid of the presently disclosed subject matter comprises a nucleotide sequence that is less than 100% identical to a target sequence (for example, is at least 60%, 65%, 60%, 65%, 70%, 75%, 80%, 85%, 90%, or 95% identical to a target sequence present in a target cell).

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between an inhibitory nucleic acid and a target sequence and embraces minor mismatches that can be accommodated by reducing the stringency of the hybridization media to achieve the desired hybridization. In some embodiments, an inhibitory nucleic acid and a target sequence hybridize substantially to each other in vivo inside of a cell.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes. Elsevier, New York, N.Y., United States of America. Generally, highly stringent hybridization and wash conditions are selected to be about 5° C. lower than the thermal melting point (Tm) for the specific sequence at a defined ionic strength and pH. Typically, under “stringent conditions” a probe will hybridize specifically to its target subsequence, but to no other sequences.

The Tm is the temperature (under defined ionic strength and pH) at which 50% of the target sequence hybridizes to a perfectly matched probe. Very stringent conditions are selected to be equal to the Tm for a particular probe. An example of highly stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1× standard saline citrate (SSC), 0.1% (w/v) SDS at 65° C. Another example of highly stringent wash conditions is 15 minutes in 0.2×SSC buffer at 65° C. (see Sambrook & Russell, 2001 for a description of SSC buffer and other stringency conditions). Often, a high stringency wash is preceded by a lower stringency wash to remove background probe signal. An example of medium stringency wash conditions for a duplex of more than about 100 nucleotides is 15 minutes in 1×SSC at 45° C. Another example of medium stringency wash for a duplex of more than about 100 nucleotides is 15 minutes in 4-6×SSC at 40° C. For short probes (e.g., about 10 to 50 nucleotides), stringent conditions typically involve salt concentrations of less than about 1M Na+ ion, typically about 0.01 to 1M Na+ ion concentration (or other salts) at pH 7.0-8.3, and the temperature is typically at least about 30° C. Stringent conditions can also be achieved with the addition of destabilizing agents such as formamide. In general, a signal to noise ratio of 2-fold or higher than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO4, 1 mm EDTA at 50° C. followed by washing in 2×SSC, 0.1% SDS at 50° C.; in another example, a probe and target sequence hybridize in 7% sodium dodecyl sulfate (SDS), 0.5M NaPO4, 1 mm EDTA at 50° C. followed by washing in 1×SSC, 0.1% SDS at 50° C.; in another example, a probe and target sequence hybridize in 7% sodium dodecyl sulfate (SDS), 0.5M NaPO4, 1 mm EDTA at 50° C. followed by washing in 0.5×SSC, 0.1% SDS at 50° C.; in another example, a probe and target sequence hybridize in 7% sodium dodecyl sulfate (SDS), 0.5M NaPO4, 1 mm EDTA at 50° C. followed by washing in 0.1×SSC, 0.1% SDS at 50° C.; in yet another example, a probe and target sequence hybridize in 7% sodium dodecyl sulfate (SDS), 0.5M NaPO4, 1 mm EDTA at 50° C. followed by washing in 0.1×SSC, 0.1% SDS at 65° C.

The term “subsequence” refers to a sequence of nucleic acids that comprises a part of a longer nucleic acid sequence. An exemplary subsequence is a sequence that comprises part of a duplexed region of an inhibitory nucleic acid molecule, one strand of which is complementary to the sequence of a target nucleic acid molecule, such as an mRNA.

As used herein, a first polynucleotide can be “derived from” a second polynucleotide if it has the same or substantially the same nucleotide sequence as a region of the second polynucleotide, its cDNA, complements thereof, or if it displays sequence identity as described above. This term is not meant to require or imply the polynucleotide must be obtained from the origin cited (although such is encompassed), but rather can be made by any suitable method.

Similarly, a first polypeptide (or peptide) is “derived from” a second polypeptide (or peptide) if it is (i) encoded by a first polynucleotide derived from a second polynucleotide, or (ii) displays sequence identity to the second polypeptides as described above. This term is not meant to require or imply the polypeptide must be obtained from the origin cited (although such is encompassed), but rather can be made by any suitable method.

The phrase “in combination with” as used herein refers to uses where, for example, a first therapy is administered during the entire course of administration of a second therapy; where the first therapy is administered for a period of time that is overlapping with the administration of the second therapy, e.g. where administration of the first therapy begins before the administration of the second therapy and the administration of the first therapy ends before the administration of the second therapy ends; where the administration of the second therapy begins before the administration of the first therapy and the administration of the second therapy ends before the administration of the first therapy ends; where the administration of the first therapy begins before administration of the second therapy begins and the administration of the second therapy ends before the administration of the first therapy ends; where the administration of the second therapy begins before administration of the first therapy begins and the administration of the first therapy ends before the administration of the second therapy ends. As such, “in combination” can also refer to regimen involving administration of two or more therapies. “In combination with” as used herein also refers to administration of two or more therapies which can be administered in the same or different formulations, by the same or different routes, and in the same or different dosage form type.

The terms “treatment”, “treating”, “treat”, and the like, refer to obtaining a desired pharmacologic and/or physiologic effect. The effect can in some embodiments be prophylactic in terms of completely or partially preventing a disease and/or a symptom thereof and/or can be therapeutic in terms of a partial or complete cure for a disease and/or adverse effect attributable to the disease. As used herein, “treatment” covers any treatment of a disease in a mammal, particularly in a human, and includes: (a) preventing the disease from occurring in a subject which might be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a penetrating peptide composition that can elicit an immune response or confer immunity in the absence of a disease condition, e.g., in the case of a vaccine.

The terms “subject” and “subject” are used interchangeably herein to refer to an animal, human or non-human, amenable to therapy according to the methods of the disclosure or to which a peptide composition according to the present disclosure can be administered to achieve a desired effect. Generally, a subject is a mammalian subject, optionally a human.

As used herein, the term “dermatitis” refers to inflammation of the skin and includes but is not limited to allergic contact dermatitis, urticaria, asteatotic dermatitis (dry skin on the lower legs), atopic dermatitis, contact dermatitis including irritant contact dermatitis and urushiol-induced contact dermatitis, eczema, gravitational dermatitis, nummular dermatitis, otitis externa, perioral dermatitis, and seborrhoeic dermatitis.

The phrase “stratum corneum” refers to the horny outer layer of the epidermis, consisting of several layers of flat, keratinized, nonnucleated, dead or peeling cells.

As used herein, the term “comprising”, which is synonymous with “including”, “containing”, and “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, and/or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, the peptides of the presently disclosed subject matter in some embodiments can “consist essentially of” a core amino acid sequence, which means that the peptide can include one or more (e.g., 1, 2, 3, 4, 5, 6, or more) N-terminal and/or C-terminal amino acids the presence of which does not materially affect the desired biological activity of the peptide.

With respect to the terms “comprising”, “consisting essentially of”, and “consisting of”, where one of these three terms is used herein, the presently disclosed subject matter can include the use of either of the other two terms. For example, the presently disclosed subject matter relates in some embodiments to compositions comprising the amino acid sequence HIITDPNMAEYL (SEQ ID NO:1). It is understood that the presently disclosed subject matter thus also encompasses peptides that in some embodiments consist essentially of the amino acid sequence HIITDPNMAEYL (SEQ ID NO:1); as well as peptides that in some embodiments consist of the amino acid sequence HIITDPNMAEYL (SEQ ID NO:1). Similarly, it is also understood that the methods of the presently disclosed subject matter in some embodiments comprise the steps that are disclosed herein and/or that are recited in the claims, that they in some embodiments consist essentially of the steps that are disclosed herein and/or that are recited in the claims, and that they in some embodiments consist of the steps that are disclosed herein and/or that are recited in the claim.

### III. Peptides

In some embodiments, the instant disclosure is directed to peptides that both alone and when conjugated to and/or associated with an active agent and/or an active agent carrier are capable of penetrating the SC and/or the cellular membranes of viable cells such as epidermal and dermal cells. Related compositions and methods are also described herein.

III.A. Skin Penetrating Peptides (SPPs)

The present disclosure provides peptides that are capable of penetrating the SC and/or penetrating viable cells following administration. These peptides are referred to herein as “penetrating peptides” or “skin penetrating peptides (SPPs)”. In some embodiments, these penetrating peptides are capable of penetrating the cellular membranes of viable epidermal and dermal cells. Penetrating peptides according to the present disclosure can comprise, consist essentially of, or consist of, for example, one or more of the amino acid sequences provided in Table 2 below.

TABLE 2
Exemplary Penetrating Peptides

| Core Peptide | SEQ | Core Peptide | SEQ |
|---|---|---|---|
| Sequence | ID NO. | Sequence | ID NO. |
| HIITDPNMAEYL | 1 | AYNAGSILENNF | 5 |
| SYTQRADSTTLH | 2 | LVPDRMTAISRA | 6 |
| GYGFSNTNSFFV | 3 | NSLRNYDFLITM | 7 |
| SHMQNRPASDEH | 4 | | |

In some embodiments, penetrating peptides according to the present disclosure include an amino acid sequence including a stretch of three, four, five, six, or seven consecutive amino acids selected from one of the following amino acid sequences: HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7).

In some embodiments, penetrating peptides according to the present disclosure have an amino acid sequence from 8 to 11, 12 to 15, or 16 to 19 amino acids in length, including an amino acid sequence selected from one of SEQ ID NOs: 1-7. In some embodiments, penetrating peptides according to the present disclosure have an amino acid sequence of at least 10, at least 20, at least 30, at least 40, at least 50, at least 60, at least 70, at least 80, at least 90, or at least 100 amino acids, wherein the amino acid sequence comprises an amino acid sequence as set forth in any of SEQ ID NOs: 1-7.

In some embodiments, a penetrating peptide of the presently disclosed subject matter is circularized. The presently disclosed penetrating peptides can be circularized by any of a variety of known cross-linking methods. In some embodiments, a penetrating peptide according to the present disclosure is provided in a circularized conformation (i.e., as a cyclic peptide) in which a Cys-Cys disulfide bond is present. In some embodiments, penetrating peptides according to the present disclosure have an amino acid sequence that comprises an internal amino acid sequence selected from one or more of SEQ ID NOs: 1-7, wherein the amino acid sequence of the peptide further comprises at least a first Cys positioned external to the internal sequence in the N-terminal direction and at least a second Cys positioned external to the internal sequence in the C-terminal direction (i.e., include a Cys residue added to the N-terminus and/or the C-terminus of any of SEQ ID NOs: 1-7. In the case where the peptides of SEQ ID NOs: 1-7 are multimerized (either the same peptide or a plurality of peptides multimerized head to tail, optionally with one or more intervening amino acids between the individual peptide sequences), the multimerized “poly”peptide can include at least two Cys residues interspersed therein to allow for circularization. In some embodiments, a first Cys residue is present at or near the N-terminus and a second Cys residue is present at or near the C-terminus of the multimerized “poly”peptide.

In some embodiments, penetrating peptides according to the present disclosure include an amino acid sequence including an internal stretch of three, four, five, or six consecutive amino acids selected from one of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7); and further including at least a first Cys positioned external to the internal sequence in the N-terminal direction and at least a second Cys positioned external to the internal sequence in the C-terminal direction. In some embodiments, a Cys residue is added to a peptide or polypeptide sequence by adding an Ala-Cys dipeptide at or near the N-terminus and/or a Cys-Gly dipeptide at or near the C-terminus. Thus, the presently disclosed subject matter also includes peptides based on SEQ ID NOs: 1-7 that have an AC dipeptide at or near the N-terminus and/or a GC dipeptide at or near the C-terminus.

The penetrating peptides disclosed herein include those having the amino acid sequences provided, as well as peptides having one or more amino acid substitutions, e.g., one or more conservative amino acid substitutions, relative to the sequences provided, wherein the peptides retains the capability of penetrating the SC or penetrating a cell. Conservative amino acid substitutions, such as those which might be employed in modifying the penetrating peptides described herein, are generally based on the relative similarity of the amino acid side-chain substituents. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Other biologically functionally equivalent changes will be appreciated by those of skill in the art.

In making perpetrating peptides with amino acid substitutions derived from SEQ ID NOs: 1-7, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (see e.g., Kyte & Doolittle (1982) J Mol Biol 157:105-132, herein incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within in some embodiments ±2 of the original value, within in some embodiments ±1 of the original value, and within in some embodiments ±0.5 of the original value can be selected.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydropathic indices are within in some embodiments ±2 of the original value, within in some embodiments ±1 of the original value, and within in some embodiments ±0.5 of the original value can be selected.

While discussion has focused on modifying SEQ ID NOs: 1-7 via conservative amino acid changes, it will be appreciated that these changes can be effected by alteration of the encoding DNA, taking into consideration also that the genetic code is degenerate and that two or more codons can code for the same amino acid.

III.B. Active Agents

The ability of the above peptides to penetrate the SC following topical administration and/or to penetrate the cellular membranes of viable cells, e.g., epidermal and dermal cells, while conjugated to or associated with a molecular cargo, e.g., a low molecular weight compound or macromolecule, makes them suitable for facilitating the delivery of a wide variety of active agents known in the art.

General classes of active agents which can be delivered include, for example, proteins, peptides, nucleic acids, nucleotides, nucleosides and analogues thereof; as well as pharmaceutical compounds, e.g., low molecular weight compounds.

Active agents which can be delivered using the penetrating peptides disclosed herein include agents which act at and/or on the skin, the peripheral nerves, adrenergic receptors, cholinergic receptors, the skeletal muscles, the cardiovascular system, smooth muscles, the blood circulatory system, synaptic sites, neuroeffector junction sites, endocrine and hormone systems, the immunological system, the reproductive system, the skeletal system, autacoid systems, the alimentary and excretory systems, the histamine system, and/or the central nervous system.

Suitable active agents can be selected, for example, from dermatological agents, anti-neoplastic agents, cardiovascular agents, renal agents, gastrointestinal agents, rheumatologic agents, immunological agents, and neurological agents among others.

Suitable dermatological agents can include, for example, local anesthetics, anti-inflammatory agents, anti-infective agents, agents to treat acne, anti-virals, anti-fungals, agents for psoriasis such as topical corticosteroids among others.

In some embodiments, a suitable dermatological agent is selected from the following list: 16-17A-Epoxyprogesterone (CAS Registry Number: 1097-51-4), P-methoxycinnamic acid/4-Methoxycinnamic acid (CAS Registry Number: 830-09-1), Octyl Methoxycinnamate (CAS Registry Number: 5466-77-3), Octyl Methoxycinnamate (CAS Registry Number: 5466-77-3), Methyl p-methoxy cinnamate (CAS Registry Number: 832-01-9), 4-ESTREN-17β-OL-3-ONE (CAS Registry Number: 62-90-8), Ethyl-p-anisoyl acetate (CAS Registry Number: 2881-83-6), Dihydrouracil (CAS Registry Number: 1904-98-9), Lopinavir (CAS Registry Number: 192725-17-0), RITANSERIN(CAS Registry Number: 87051-43-2), Nilotinib (CAS Registry Number: 641571-10-0); Rocuronium bromide (CAS Registry Number: 119302-91-9), p-Nitrobenzyl-6-(1-hydroxyethyl)-1-azabicyclo(3.2.0)heptane-3,7-dione-2-carboxylate (CAS Registry Number: 74288-40-7), Abamectin (CAS Registry Number: 71751-41-2), Paliperidone (CAS Registry Number: 144598-75-4), Gemifioxacin (CAS Registry Number: 175463-14-6), Valrubicin (CAS Registry Number: 56124-62-0), Mizoribine (CAS Registry Number: 50924-49-7), Solifenacin succinate (CAS Registry Number: 242478-38-2), Lapatinib (CAS Registry Number: 231277-92-2), Dydrogesterone (CAS Registry Number: 152-62-5), 2,2-Dichloro-N-[(1R,2S)-3-fluoro-1-hydroxy-1-(4-methylsulfonylphenyl)propan-2-yl]acetamide (CAS Registry Number: 73231-34-2), Tilmicosin (CAS Registry Number: 108050-54-0), Efavirenz (CAS Registry Number: 154598-52-4), Pirarubicin (CAS Registry Number: 72496-41-4), Nateglinide (CAS Registry Number: 105816-04-4), Epirubicin (CAS Registry Number: 56420-45-2), Entecavir (CAS Registry Number: 142217-69-4), Etoricoxib (CAS Registry Number: 202409-33-4), Cilnidipine (CAS Registry Number: 132203-70-4), Doxorubicin hydrochloride (CAS Registry Number: 25316-40-9), Escitalopram (CAS Registry Number: 128196-01-0), Sitagliptin phosphate monohydrate (CAS Registry Number: 654671-77-9), Acitretin (CAS Registry Number: 55079-83-9), Rizatriptan benzoate (CAS Registry Number: 145202-66-0), Doripenem (CAS Registry Number: 148016-81-3), Atracurium besylate (CAS Registry Number: 64228-81-5), Nilutamide (CAS Registry Number: 63612-50-0), 3,4-Dihydroxyphenylethanol (CAS Registry Number: 10597-60-1), KETANSERIN TARTRATE (CAS Registry Number: 83846-83-7), Ozagrel (CAS Registry Number: 82571-53-7), Eprosartan mesylate (CAS Registry Number: 144143-96-4), Ranitidine hydrochloride (CAS Registry Number: 66357-35-5), 6,7-Dihydro-6-mercapto-5H-pyrazolo[1,2-a][1,2,4]triazolium chloride (CAS Registry Number: 153851-71-9), Sulfapyridine (CAS Registry Number: 144-83-2), Teicoplanin (CAS Registry Number: 61036-62-2), Tacrolimus (CAS Registry Number: 104987-11-3), LUMIRACOXIB (CAS Registry Number: 220991-20-8), Allyl alcohol (CAS Registry Number: 107-18-6), Protected meropenem (CAS Registry Number: 96036-02-1), Nelarabine (CAS Registry Number: 121032-29-9), Pimecrolimus (CAS Registry Number: 137071-32-0), 4-[6-Methoxy-7-(3-piperidin-1-ylpropoxy)quinazolin-4-yl]-N-(4-propan-2-yloxyphenyl)piperazine-1-carboxamide (CAS Registry Number: 387867-13-2), Ritonavir (CAS Registry Number: 155213-67-5), Adapalene (CAS Registry Number: 106685-40-9), Aprepitant (CAS Registry Number: 170729-80-3), Eplerenone (CAS Registry Number: 107724-20-9), Rasagiline mesylate (CAS Registry Number: 161735-79-1), Miltefosine (CAS Registry Number: 58066-85-6), Raltegravir potassium (CAS Registry Number: 871038-72-1), Dasatinib monohydrate (CAS Registry Number: 863127-77-9), OXOMEMAZINE (CAS Registry Number: 3689-50-7), Pramipexole (CAS Registry Number: 104632-26-0), PARECOXIB SODIUM (CAS Registry Number: 198470-85-8), Tigecycline (CAS Registry Number: 220620-09-7), Toltrazuril (CAS Registry Number: 69004-03-1), Vinflunine (CAS Registry Number: 162652-95-1), Drospirenone (CAS Registry Number: 67392-87-4), Daptomycin (CAS Registry Number: 103060-53-3), Montelukast sodium (CAS Registry Number: 151767-02-1), Brinzolamide (CAS Registry Number: 138890-62-7), Maraviroc (CAS Registry Number: 376348-65-1), Doxercalciferol (CAS Registry Number: 54573-75-0), Oxolinic acid (CAS Registry Number: 14698-29-4), Daunorubicin hydrochloride (CAS Registry Number: 23541-50-6), Nizatidine (CAS Registry Number: 76963-41-2), Idarubicin (CAS Registry Number: 58957-92-9), FLUOXETINE HYDROCHLORIDE (CAS Registry Number: 59333-67-4), Ascomycin (CAS Registry Number: 11011-38-4), beta-Methyl vinyl phosphate (MAP) (CAS Registry Number: 90776-59-3), Amorolfine (CAS Registry Number: 67467-83-8), Fexofenadine HCl (CAS Registry Number: 83799-24-0), Ketoconazole (CAS Registry Number: 65277-42-1), 9,10-difluoro-2,3-dihydro-3-me-7-oxo-7H-pyrido-1 (CAS Registry Number: 82419-35-0), Ketoconazole (CAS Registry Number: 65277-42-1), Terbinafine HCl (CAS Registry Number: 78628-80-5), Amorolfine (CAS Registry Number: 78613-35-1), Methoxsalen (CAS Registry Number: 298-81-7), Olopatadine HCl (CAS Registry Number: 113806-05-6), Zinc Pyrithione (CAS Registry Number: 13463-41-7), Olopatadine HCl (CAS Registry Number: 140462-76-6), Cyclosporine (CAS Registry Number: 59865-13-3), and Botulinum toxin and its analogs and vaccine components.

III.B.1. Proteins, Polypeptides, and Peptides as Active Agents

Proteins useful in the disclosed depot formulations can include, for example, molecules such as cytokines and their receptors, as well as chimeric proteins including cytokines or their receptors, including, for example tumor necrosis factor alpha and beta, their receptors and their derivatives; renin; growth hormones, including human growth hormone, bovine growth hormone, methione-human growth hormone, des-phenylalanine human growth hormone, and porcine growth hormone; growth hormone releasing factor (GRF); parathyroid and pituitary hormones; thyroid stimulating hormone; human pancreas hormone releasing factor; lipoproteins; colchicine; prolactin; corticotrophin; thyrotropic hormone; oxytocin; vasopressin; somatostatin; lypressin; pancreozymin; leuprolide; alpha-1-antitrypsin; insulin A-chain; insulin B-chain; proinsulin; follicle stimulating hormone; calcitonin; luteinizing hormone; luteinizing hormone releasing hormone (LHRH); LHRH agonists and antagonists; glucagon; clotting factors such as factor VIIIC, factor IX, tissue factor, and von Willebrands factor; anti-clotting factors such as Protein C; atrial natriuretic factor; lung surfactant; a plasminogen activator other than a tissue-type plasminogen activator (t-PA), for example a urokinase; bombesin; thrombin; hemopoietic growth factor; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; gonadotropin releasing hormone; bovine somatotropin; porcine somatotropin; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, 4, -5, or -6 (NT-3, NT-4, NT-5, or NT-6), or a nerve growth factor such as NGF-13; platelet-derived growth factor (PDGF); fibroblast growth factor such as acidic FGF and basic FGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha (e.g., interferonα2A), -beta, -gamma, -lambda and consensus interferon; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV-1 envelope glycoprotein, gp120, gp160 or fragments thereof; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues.

Suitable proteins or peptides can be native or recombinant and include, e.g., fusion proteins.

In some embodiments, the protein is a growth hormone, such as human growth hormone (hGH), recombinant human growth hormone (rhGH), bovine growth hormone, methione-human growth hormone, des-phenylalanine human growth hormone, and porcine growth hormone; insulin, insulin A-chain, insulin B-chain, and proinsulin; or a growth factor, such as vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

Suitable peptides for use as the active agent in the injectable, biodegradable delivery depots disclosed herein include, but are not limited to, Glucagon-like peptide-1 (GLP-1) and precursors, derivatives, prodrugs and analogues thereof.

III.B.2. Nucleic Acids as Active Agents Nucleic acid active agents include nucleic acids as well as precursors, derivatives, prodrugs and analogues thereof, e.g., therapeutic nucleotides, nucleosides and analogues thereof; therapeutic oligonucleotides; and therapeutic polynucleotides. Active agents selected from this group can find particular use as anticancer agents and antivirals. Suitable nucleic acid active agents can include for example ribozymes, antisense oligodeoxynucleotides, aptamers and siRNA. Examples of suitable nucleoside analogues include, but are not limited to, cytarabine (araCTP), gemcitabine (dFdCTP), and floxuridine (FdUTP). In some embodiments, a suitable nucleic acid active agent is an interfering RNA, e.g., shRNA, miRNA or siRNA. Suitable siRNAs include, for example, IL-7 (Interleukin-7) siRNA, IL-10 (Interleukin-10) siRNA, IL-22 (Interleukin-22) siRNA, IL-23 (Interleukin 23) siRNA, CD86 siRNA, KRT6a (keratin 6A) siRNA, K6a N171K (keratin 6a N171K) siRNA, TNFα (tumor necrosis factor α) siRNA, TNFR1 (tumor necrosis factor receptor-1) siRNA, TACE (tumor necrosis factor (TNF)-α converting enzyme) siRNA, RRM2 (ribonucleotide reductase subunit-2) siRNA, and VEGF (vascular endothelial growth factor) siRNA. mRNA sequences of the human gene targets of these siRNAs are known in the art. For IL-7, see, e.g., GENBANK® Accession: NM_000880.3, GENBANK® Accession No. NM_001199886.1, GENBANK® Accession No. NM_001199887.1, and GENBANK® Accession No. NM_001199888.1; for IL-10, see, e.g., GENBANK® Accession No. NM_000572.2; for IL-22 see, e.g., GENBANK® Accession No. NM_020525.4; for IL-23, see, e.g., GENBANK® Accession No. NM_016584.2, and GENBANK® Accession No. AF301620.1; for CD86, see, e.g., GENBANK® Accession No. NM_175862.4, GENBANK® Accession No. NM_006889.4, GENBANK® Accession No. NM_176892.1, GENBANK® Accession No. NM_001206924.1, and GENBANK® Accession No. NM_001206925.1; for KRT6a, see, e.g., GENBANK® Accession No. NM_005554.3; for TNFα, see, e.g., GENBANK® Accession No. NM_000594.2; for TNFR1, see, e.g., GENBANK® Accession No. NM_001065.3; for TACE, see, e.g., GENBANK® Accession No. NM_003183.4; for RRM2, see, e.g., GENBANK® Accession No. NM_001165931.1 and GENBANK® Accession No. NM_001034.3; for VEGF, see, e.g., GENBANK® Accession No. NM_001025366.2, GENBANK® Accession No. NM_001025367.2, GENBANK® Accession No. NM_001025368.2, GENBANK® Accession No. NM_001025369.2, GENBANK® Accession No. NM_001025370.2, NM_001033756.2, GENBANK® Accession No. NM_001171622.1, and GENBANK® Accession No. NM_003376.5.

In addition a variety of methods and techniques are known in the art for selecting a particular mRNA target sequence during siRNA design. See e.g., the publicly available siRNA WIZARD™ design tool provided on the Internet by InvivoGen of San Diego, Calif., United States of America.

III.B.3. Additional Active Agent Compounds

A variety of additional active agent compounds can be used in the injectable depot compositions disclosed herein. Suitable compounds can include compounds directed to one or more of the following drug targets: Kringle domain, Carboxypeptidase, Carboxylic ester hydrolases, Glycosylases, Rhodopsin-like dopamine receptors, Rhodopsin-like adrenoceptors, Rhodopsin-like histamine receptors, Rhodopsin-like serotonin receptors, Rhodopsin-like short peptide receptors, Rhodopsin-like acetylcholine receptors, Rhodopsin-like nucleotide-like receptors, Rhodopsin-like lipid-like ligand receptors, Rhodopsin-like melatonin receptors, Metalloprotease, Transporter ATPase, Carboxylic ester hydrolases, Peroxidase, Lipoxygenase, DOPA decarboxylase, A/G cyclase, Methyltransferases, Sulphonylurea receptors, other transporters (e.g., Dopamine transporter, GABA transporter 1, Norepinephrine transporter, Potassium-transporting ATPase α-chain 1, Sodium-(potassium)-chloride cotransporter 2, Serotonin transporter, Synaptic vesicular amine transporter, and Thiazide-sensitive sodium-chloride cotransporter), Electrochemical nucleoside transporter, Voltage-gated ion channels, GABA receptors (Cys-Loop), Acetylcholine receptors (Cys-Loop), NMDA receptors, 5-HT3 receptors (Cys-Loop), Ligand-gated ion channels Glu: kainite, AMPA Glu receptors, Acid-sensing ion channels aldosterone, Ryanodine receptors, Vitamin K epoxide reductase, MetGluR-like GABA_{B} receptors, Inwardly rectifying K⁺ channel, NPC1L1, MetGluR-like calcium-sensing receptors, Aldehyde dehydrogenases, Tyrosine 3-hydroxylase, Aldose reductase, Xanthine dehydrogenase, Ribonucleoside reductase, Dihydrofolate reductase, IMP dehydrogenase, Thioredoxin reductase, Dioxygenase, Inositol monophosphatase, Phosphodiesterases, Adenosine deaminase, Peptidylprolyl isomerases, Thymidylate synthase, Aminotransferases, Farnesyl diphosphate synthase, Protein kinases, Carbonic anhydrase, Tubulins, Troponin, Inhibitor of IκB kinase-β, Amine oxidases, Cyclooxygenases, Cytochrome P450s, Thyroxine 5-deiodinase, Steroid dehydrogenase, HMG-CoA reductase, Steroid reductases, Dihydroorotate oxidase, Epoxide hydrolase, Transporter ATPase, Translocator, Glycosyltransferases, Nuclear receptors NR3 receptors, Nuclear receptors: NR1 receptors, and Topoisomerase.

In some embodiments, the active agent is a compound targeting one of rhodopsin-like GPCRs, nuclear receptors, ligand-gated ion channels, voltage-gated ion channels, penicillin-binding protein, myeloperoxidase-like, sodium: neurotransmitter symporter family, type II DNA topoisomerase, fibronectin type III, and cytochrome P450.

In some embodiments, the active agent is an anticancer agent. Suitable anticancer agents include, but are not limited to, Actinomycin D, Alemtuzumab, Allopurinol sodium, Amifostine, Amsacrine, Anastrozole, Ara-CMP, Asparaginase, Azacytadine, Bendamustine, Bevacizumab, Bicalutimide, Bleomycin (e.g., Bleomycin A₂ and B₂), Bortezomib, Busulfan, Camptothecin sodium salt, Capecitabine, Carboplatin, Carmustine, Cetuximab, Chlorambucil, Cisplatin, Cladribine, Clofarabine, Cyclophosphamide, Cytarabine, Dacarbazine, Dactinomycin, Daunorubicin, Daunorubicin liposomal, Dacarbazine, Decitabine, Docetaxel, Doxorubicin, Doxorubicin liposomal, Epirubicin, Estramustine, Etoposide, Etoposide phosphate, Exemestane, Floxuridine, Fludarabine, Fludarabine phosphate, 5-Fluorouracil, Fotemustine, Fulvestrant, Gemcitabine, Goserelin, Hexamethylmelamine, Hydroxyurea, Idarubicin, Ifosfamide, Imatinib, Irinotecan, Ixabepilone, Lapatinib, Letrozole, Leuprolide acetate, Lomustine, Mechlorethamine, Melphalan, 6-Mercaptopurine, Methotrexate, Mithramycin, Mitomycin C, Mitotane, Mitoxantrone, Nimustine, Ofatumumab, Oxaliplatin, Paclitaxel, Panitumumab, Pegaspargase, Pemetrexed, Pentostatin, Pertuzumab, Picoplatin, Pipobroman, Plerixafor, Procarbazine, Raltitrexed, Rituximab, Streptozocin, Temozolomide, Teniposide, 6-Thioguanine, Thiotepa, Topotecan, Trastuzumab, Treosulfan, Triethylenemelamine, Trimetrexate, Uracil Nitrogen Mustard, Valrubicin, Vinblastine, Vincristine, Vindesine, Vinorelbine, and analogues, precursors, derivatives and pro-drugs thereof. It should be noted that two or more of the above compounds can be used in combination in the penetrating peptide compositions of the present disclosure.

Active agents of interest for use in the disclosed penetrating peptide compositions can also include opioids and derivatives thereof as well as opioid receptor agonists and antagonists, e.g., naltrexone, naloxone, nalbuphine, fentanyl, sufentanil, oxycodone, and pharmaceutically acceptable salts and derivatives thereof.

In some embodiments the active agent is a small molecule or low molecular weight compound, e.g., a molecule or compound having a molecular weight of less than or equal to about 1000 Daltons, e.g., less than or equal to about 800 Daltons.

In some embodiments, the active agent is a label. Suitable labels include, e.g, radioactive isotopes, fluorescers, chemiluminescers, chromophores, enzymes, enzyme substrates, enzyme cofactors, enzyme inhibitors, chromophores, dyes, metal ions, magnetic particles, nanoparticles and quantum dots.

The active agent can be present in any suitable concentration in the compositions disclosed herein. Suitable concentrations can vary depending on the potency of the active agent, active agent half-life, etc. In addition, penetrating peptide compositions according to the present disclosure can include one or more active agents, e.g., a combination of two or more of the active agents described above.

III.C. Active Agent Carriers

As described previously herein one or more active agents can be conjugated to or associated with a penetrating peptide to provide a penetrating peptide composition according to the present disclosure. Alternatively, a penetrating peptide composition according to the present disclosure can include a penetrating peptide as disclosed herein conjugated or associated with an active agent carrier which in turn includes the active agent attached thereto and/or disposed therein.

Suitable active agent carriers include, for example, liposomes, nanoparticles, micelles, microbubbles, and the like. Techniques for incorporating active agents into such carriers are known in the art. For example, liposomes or lipidic particles can be prepared in accordance with U.S. Pat. No. 5,077,057 to Szoka, Jr. Liposomes formed from nonphosphal lipid components which have the potential to form lipid bilayers are disclosed in Brockerhoff & Ramsammy (1982) Biochim Biophys Acta—Membranes 19:227-232. For the preparation, purification, modification, and loading of liposomes, see generally New (1990) Liposomes: A Practical Approach, Oxford University Press Inc., New York, N.Y., United States of America.

A general discussion of techniques for preparation of liposomes and of medication encapsulating liposomes can be found in U.S. Pat. No. 4,224,179 to Schneider. See also Mayer et al. (1986) Chemistry and Physics of Lipids 40:333-345. See also U.S. Pat. No. 6,083,539 to Yamada & Iljima for the encapsulation of an active agent dry powder composition. For incorporation of active agents into nanoparticles, see e.g., de Villiers et al. (eds) (2009) Nanotechnology in Drug Delivery, American Association of Pharmaceutical Scientists Press, Springer, New York, N.Y., United States of America. For incorporation of active agents into micelles, see e.g., Lu & Oie (2004) Cellular Drug Delivery: Principles and Practice, Humana Press Inc., Totowa, N.J., United States of America.

III.D. Attachment of Peptides to Active Agents and Active Agent Carriers

Penetrating peptides as described herein can be conjugated to or associated with an active agent. Alternatively, a penetrating peptide as disclosed herein can conjugated or associated with an active agent carrier, which in turn includes the active agent attached thereto and/or disposed therein (examples of which are discussed above). Conjugation techniques generally result in the formation of one or more covalent bonds between the penetrating peptide and either the active agent or an active agent carrier while association techniques generally utilize one or more of hydrophobic, electrostatic or van der Walls interactions.

A variety of techniques can be used for conjugating or associating a peptide to an active agent. Similarly, a variety of techniques can be used for conjugating or associating a peptide to an active agent carrier, e.g., liposomes, nanoparticles, or micelle as described herein.

For example, where the active agent is a peptide or polypeptide, the entire composition, including the penetrating peptide, can be synthesized using standard amino acid synthesis techniques. Other methods including standard molecular biology techniques can be used to express and purify the entire polypeptide sequence including the penetrating peptide. Additional methods of conjugating peptides to other peptides or polypeptides include Cu-catalyzed azide/alkyne [3+2] cycloaddition “Click Chemistry” as described by Rostovtsev et al. (2002) Angew Chem Int Ed. 41:2596-2599 and Tornoe et al. (2002) J Org Chem 67:3057-3064; azide/DIFO (Difluorinated Cyclooctyne) Cu-free Click Chemistry as described by Baskin et al. (2007) Proc Natl Acad Sci USA 104:16793-16797; azide/phosphine “Staudinger Reaction” as described by Lin et al. (2005) J Am Chem Soc 127:2686-2695; azide/triarylphosphine “Modified Staudinger Reaction” as described by Saxon & Bertozzi (2000) Science 287:2007-2010; and catalyzed olefin cross metathesis reactions as described by Casey (2006) J Chem Edu 83:192-195; Lynn et al. (2000) J Am Chem Soc 122:6601-6609; and Chen et al. (2003) Prog Chem 15:401-408.

Where the active agent is a low molecular weight compound or small molecule, a variety of techniques can be utilized to conjugate the low molecular weight compound or small molecule to a penetrating peptide as described herein. See e.g., click chemistry as described in Loh et al. (2010) Chem Commun 46:8407-8409. See also Thomson (2004) Methods Mol Med 94:255-265, describing conjugation of small molecule carboxyl, hydroxyl, and amine residues to amine and sulfhydryl residues on proteins.

Methods are also available in the art for conjugating peptides to active agent carriers such as liposomes. See e.g., Gregoriadis (ed) (2007) Liposome Technology Third Edition, Volume II Entrapment of Drugs and Other Materials into Liposomes, Informa Healthcare, New York, N.Y., United States of America, which describes techniques for coupling peptides to the surface of liposomes. For the covalent attachment of proteins to liposomes, see e.g., New (1990) Liposomes: A Practical Approach, Oxford University Press Inc., New York, N.Y., United States of America. at pages 163-182.

III.E. Administration of Penetrating Peptide Compositions as Pharmaceutical Formulations

One skilled in the art will appreciate that a variety of suitable methods of administering a penetrating peptide composition to a subject or host, e.g., subject, in need thereof, are available, and, although more than one route can be used to administer a particular composition, a particular route can provide a more immediate and more effective reaction than another route. Pharmaceutically acceptable excipients are also well known to those who are skilled in the art, and are readily available. The choice of excipient will be determined in part by the particular compound, as well as by the particular method used to administer the composition. Accordingly, there are a wide variety of suitable formulations of the penetrating peptide compositions. The following methods and excipients are merely exemplary and are in no way limiting.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; (d) suitable emulsions and (e) hydrogels. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are known in the art.

Penetrating peptide formulations can be made into aerosol formulations to be administered via inhalation. These aerosol formulations can be placed into pressurized acceptable propellants, such as dichlorodifluoromethane, propane, nitrogen, and the like. They can also be formulated as pharmaceuticals for non-pressured preparations such as for use in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration can be presented as creams, gels, pastes, patches, sprays or foams.

Suppository formulations are also provided by mixing with a variety of bases such as emulsifying bases or water-soluble bases. Formulations suitable for vaginal administration can be presented as pessaries, tampons, creams, gels, pastes, foams.

Unit dosage forms for oral or rectal administration such as syrups, elixirs, and suspensions can be provided wherein each dosage unit, for example, teaspoonful, tablespoonful, tablet or suppository, contains a predetermined amount of the composition. Similarly, unit dosage forms for injection or intravenous administration can comprise the penetrating peptides in a formulation as a solution in sterile water, normal saline or another pharmaceutically acceptable carrier.

The term “unit dosage form”, as used herein, refers to physically discrete units suitable as unitary dosages for human and animal subjects, each unit containing a predetermined quantity of penetrating peptide composition calculated in an amount sufficient to produce the desired effect in association with a pharmaceutically acceptable diluent, carrier or vehicle. The specifications for the novel unit dosage forms of the penetrating peptide compositions depend on the particular active agent employed and the effect to be achieved, and the pharmacodynamics associated with each compound in the host.

Those of skill in the art will readily appreciate that dose levels can vary as a function of the specific compound, the nature of the delivery vehicle, and the like. Suitable dosages for a given compound are readily determinable by those of skill in the art by a variety of means.

Optionally, the pharmaceutical composition can contain other pharmaceutically acceptable components, such a buffers, surfactants, antioxidants, viscosity modifying agents, preservatives and the like. Each of these components is well-known in the art. See e.g., U.S. Pat. No. 5,985,310 to Castillo & Espino, the disclosure of which is herein incorporated by reference.

Other components suitable for use in penetrating peptide formulations can be found in Remington's Pharmaceutical Sciences, 18th edition (June 1995), Mack Publishing Co., Easton, Pa., United States of America. In some embodiments, the aqueous cyclodextrin solution further comprise dextrose, e.g., about 5% dextrose.

III.F. Administration of Penetrating Peptide Compositions as Medical Device Components

In some embodiments, one or more of the penetrating peptide compositions of the present disclosure can be incorporated into a medical device known in the art, for example, drug eluting stents, catheters, fabrics, cements, bandages (liquid or solid), biodegradable polymer depots and the like. In some embodiments, the medical device is an implantable or partially implantable medical device.

### IV. Methods of Treatment

In some embodiments, the presently disclosed subject matter provides methods for treating diseases and/or disorders using the compositions disclosed herein, wherein the compositions comprise an effective amount of a penetrating peptide composition disclosed herein. In some embodiments, an effective amount of a penetrating peptide composition disclosed herein comprises a therapeutically effective amount of a therapeutic molecule.

The terms “an effective amount” (or, in the context of a therapy, a “pharmaceutically effective amount” or a “therapeutically effective amount”) of a penetrating peptide composition generally refers to an amount of the penetrating peptide composition that is effective to accomplish the desired therapeutic effect, e.g., in the case of a penetrating peptide-siRNA composition, an amount effective to reduce expression of the targeted mRNA by an amount effective to produce a desired therapeutic effect.

Effective amounts of penetrating peptide compositions, suitable delivery vehicles, and protocols can be determined by conventional means. For example, in the context of therapy a medical practitioner can commence treatment with a low dose of one or more penetrating peptide compositions in a subject or subject in need thereof, and then increase the dosage, or systematically vary the dosage regimen, monitor the effects thereof on the subject or subject, and adjust the dosage or treatment regimen to maximize the desired therapeutic effect. Further discussion of optimization of dosage and treatment regimens can be found in Benet et al. (1996) in Goodman & Gilman's The Pharmacological Basis of Therapeutics, Ninth Edition, Hardman et al. (eds.), McGraw-Hill, New York, N.Y., United States of America (see e.g., Chapter 1, pp. 3-27), and Bauer (1999) in Pharmacotherapy, A Pathophysiologic Approach, Fourth Edition, DiPiro et al. (eds.), Appleton & Lange, Stamford, Conn., United States of America (see e.g., Chapter 3, pp. 21-43, and the references cited therein.

The dosage levels and mode of administration will be dependent on a variety of factors such as the penetrating peptides used, the active agent, the context of use (e.g., the subject to be treated), and the like. Optimization of modes of administration, dosage levels, and adjustment of protocols, including monitoring systems to assess effectiveness are routine matters well within ordinary skill.

In some embodiments, the present disclosure provides a method of treating a subject having a dermatological disease, including: administering to the subject a pharmaceutically effective amount of a composition including a penetrating peptide as disclosed herein, wherein the peptide is conjugated to or associated with a dermatological active agent, e.g., a dermatological active agent as disclosed herein, or a dermatological active agent carrier including the active agent.

In some embodiments, the present disclosure provides a method of treating a subject having, suspected of having or susceptible to a disorder resulting at least in part from expression of an mRNA, including administering to the subject a pharmaceutically effective amount of a composition including a penetrating peptide as described herein, wherein the penetrating peptide is conjugated to or associated with an interfering RNA or an active agent carrier including an interfering RNA, e.g., an shRNA, miRNA or siRNA which targets the mRNA or a carrier including the interfering RNA.

In some embodiments, the interfering RNA is an siRNA. In some embodiments, the siRNA is designed to target a nucleic acid that encodes a polypeptide that has a biological activity that one might wish to modulate in a cell, tissue, or subject. Exemplary non-limiting classes of polypeptides that have biological activities that can be modulated with siRNAs include interleukins such as but not limited to IL-10, IL-17, IL-22, and IL-23; cell signaling molecules such as but not limited to CD86; cytokines such as but not limited to TNFα, TNFβ, and molecules associated with cytokine signaling such as but not limited to TACE and cytokine receptors.

In some embodiments, the presently disclosed subject matter provides methods for delivering active agents to subjects, the methods comprising administering to a subject at least one composition comprising at least one peptide comprising an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7), wherein at least one peptide is conjugated to and/or associated with at least one active agent or at least one active agent carrier comprising the at least one active agent, and wherein the at least one composition is capable of penetrating the stratum corneum (SC) of the subject and/or penetrating a cell of the subject.

In some embodiments, the presently disclosed subject matter also provides methods for treating a subject having a dermatological disease, the method comprising administering to the subject at least one composition comprising at least one peptide comprising an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7), wherein the at least one peptide is conjugated to and/or associated with at least one dermatologically active agent and/or at least one dermatologically active agent carrier comprising the at least one active agent, and further wherein the at least one composition is capable of penetrating the stratum corneum (SC) of the subject or penetrating a cell of the subject.

The presently disclosed subject matter also provides in some embodiments methods for treating subjects having, suspected of having, or susceptible to a disorder resulting at least in part from expression of an mRNA, comprising administering to a subject a composition comprising at least one peptide comprising at least one amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7). In some embodiments, the at least one peptide is conjugated to and/or associated with at least one interfering RNA that targets the mRNA and/or a carrier comprising the interfering RNA; the at least one composition is capable of penetrating the stratum corneum (SC) of the subject or a cell of the subject, and the administering step results in expression of the mRNA being attenuated thereby. In some embodiments, the at least one peptide is associated with at least one interfering RNA that targets the mRNA and/or at least one carrier comprising the at least one interfering RNA and the association results from hydrophobic, electrostatic, and/or van der Walls interactions between the at least one peptide and the at least one interfering RNA.

Summarily, the presently disclosed peptides and peptide composition can be employed in some embodiments as a drug delivery system to deliver small and large molecules for localized (e.g., to skin or scalp) and/or systemic drug delivery through skin; in some embodiments as a vaccine delivery system to develop an effective transcutaneous vaccine delivery system where the vaccine and adjuvant can be simultaneously delivered to the Langerhan's cells in the viable epidermis and dendritic cells in the epidermis; and in some embodiments as a gene delivery system to deliver gene based therapies alone or in combination with conventional chemotherapy (including, but not limited to for delivery of siRNA, antisense oligonucleotides, and/or anti-cancer drugs). In some embodiments, the presently disclosed peptides and peptide composition can be employed for treatment of skin cancer and/or other multifactorial skin diseases such as but not limited to psoriasis.

### V. Methods and Compositions for Inducing Immune Responses

In some embodiments, the presently disclosed subject matter provides methods and compositions for inducing immune responses.

In some embodiments, a method for inducing an immune response comprises administering to a subject a composition comprising a peptide conjugated to and/or associated with an antigen to which an immune response in the subject is desired and/or a carrier comprising the antigen, wherein the peptide comprises an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7), the composition is capable of penetrating the stratum corneum (SC) of the subject and/or penetrating a cell of the subject to deliver the antigen across the SC or into the cell, and the antigen is present in the composition in an effective amount for eliciting an immune response in the subject.

In some embodiments, a composition for inducing an immune response is a vaccine. In some embodiments, the composition comprises a peptide conjugated to and/or associated with an antigen to which an immune response in the subject is desired and/or a carrier comprising the antigen, wherein (i) the peptide comprises an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7); (ii) the antigen is present in the composition in an amount sufficient to elicit an immune response in a subject to the antigen; and (iii) the composition penetrates the stratum corneum (SC) of the subject and/or a cell of the subject to deliver the antigen to the subject's immune system. In some embodiments, composition is in a stable, dry particulate form comprising the peptide and the antigen and/or the carrier. In some embodiments, the composition further comprises an adjuvant; a stabilizer, optionally a stabilizer selected from the group consisting of a protein stabilizer, a sugar, and a sugar derivative; a pharmaceutically acceptable carrier or diluent; or any combination thereof. In some embodiments, the composition is formulated for topical administration, and/or if in lyophilized form, can be reconstituted for use in topical administration.

A composition of the presently disclosed subject matter comprises an amount of antigen that is sufficient to elicit an immune response in a subject to the antigen, referred to herein as an “effective amount”. In the context of eliciting immune responses, an “effective amount” of an antigen is that amount of antigen that is sufficient to elicit an immune response in a subject to the antigen subsequent to administering the composition to the subject. In some embodiments, the administering step is repeated one or more times, either with the same composition or with a modified composition, provided that the modified composition comprises at least the antigen and/or antigen carrier and, in some embodiments, further comprises the peptide.

In the context of eliciting an immune response in order to provide a treatment and/or a prevention of a disease or disorder, an “effective amount” of an antigen is that amount of antigen that is sufficient to elicit an immune response in a subject to the antigen and as a consequence, is sufficient to show a meaningful benefit to the subject, such as, enhanced immune response, treatment, healing, prevention, and/or amelioration of the relevant medical condition (disease, infection, or the like), and/or an increase in rate of treatment, healing, prevention, and/or amelioration of such a disease or disorder. In these contexts, an “effective amount” can also be referred to as a “therapeutically effective amount”. Furthermore, administering an “effective amount” or a “therapeutically effective amount” of a composition of the presently disclosed subject matter refers to a set of circumstances wherein the subject is treated with a composition of the presently disclosed subject matter in an amount and for a time sufficient to induce an improvement, in some embodiments a sustained improvement, in at least one indicator that reflects the severity of the disease, infection, or disorder.

As used herein, an improvement is considered “sustained” if the subject exhibits the improvement on at least two occasions separated by a period of time. The degree of improvement can be determined based, for example, on immunological data, or on signs or symptoms of a disease, infection, or disorder. Various indicators that reflect the extent of the subject's illness can be assessed for determining whether the amount and time of the treatment is sufficient. The baseline value for the chosen indicator or indicators can be established based on by examination of the subject prior to administration of the first dose of a composition of the presently disclosed subject matter, and/or is based on statistical values generated from a population of healthy subjects. If the therapeutic agent is administered to treat acute symptoms, the first dose is administered as soon as practically possible. Improvement is induced by administering one or more compositions of the presently disclosed subject matter until the subject manifests an improvement over baseline for the chosen indicator or indicators. In treating chronic conditions, this degree of improvement is in some embodiments obtained by repeatedly administering a composition of the presently disclosed subject matter over a period time, such as but not limited to, for one, two, or three months or longer, or in some embodiments indefinitely. In some embodiments a single dose can be sufficient for treating or preventing certain conditions. Treatment can be continued indefinitely at the same level or at a reduced dose or frequency, regardless of the subject's condition, if desired. Once treatment has been reduced or discontinued, it later can be resumed at the original level or at a different level if symptoms reappear.

Generally, the amount of a composition of the presently disclosed subject matter that provides an efficacious dose or therapeutically effective dose for vaccination is in some embodiments from about 1 μg or less to about 100 μg or more, in some embodiments from about 2, 3, 4, 5, 6, 7, 8, 9, 10, 15, 20, 25, 30, 35, 40, 4, 5 or 50 μg to about 55, 60, 65, 70, 75, 80, 85, 90, or 95 μg per kg body weight. In some embodiments, multiple injections administered over a period of days can be considered for therapeutic usage.

The compositions of the presently disclosed subject matter as vaccines can be administered as a single dose or in a series including one or more boosters. For example, an infant or child can receive a single dose early in life, then be administered a booster dose up to 1, 2, 3, 4, 5, 6, 7, 8, 9, 10, or more years later. The booster dose generates antibodies from primed B-cells (i.e., an anamnestic response). That is, the compositions of the presently disclosed subject matter as vaccines elicit a high primary functional antibody response in infants or children, and is capable of eliciting an anamnestic response following a booster administration, demonstrating that the protective immune response elicited by the conjugate vaccine is long-lived.

The compositions of the presently disclosed subject matter as vaccines can be formulated into liquid preparations for, for example, oral, nasal, anal, rectal, buccal, vaginal, peroral, intragastric, mucosal, perlingual, alveolar, gingival, olfactory, or respiratory mucosa administration. Suitable forms for such administration include suspensions, syrups, and elixirs. The compositions of the presently disclosed subject matter as vaccines can also be formulated for topical, parenteral, subcutaneous, intradermal, intramuscular, intraperitoneal, or intravenous administration, injectable administration, sustained release from implants, or administration by eye drops. Suitable forms for such administration include sterile suspensions and emulsions. Such vaccines can be in admixture with a suitable carrier, diluent, or excipient such as sterile water, physiological saline, glucose, and the like. The compositions of the presently disclosed subject matter as vaccines can also be lyophilized. The compositions of the presently disclosed subject matter as vaccines can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as Remington: The Science and Practice of Pharmacy, Lippincott Williams & Wilkins; 20th edition (Jun. 1, 2003) and Remington's Pharmaceutical Sciences, Mack Pub. Co.; 18^{th} and 19^{th} editions (December 1985, and June 1990, respectively), incorporated herein by reference in their entireties, can be consulted to prepare suitable preparations. Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

The vaccines of the presently disclosed subject matter are in some embodiments provided as liquid suspensions and in some embodiments are provided as freeze-dried products. Suitable liquid preparations include, for example, isotonic aqueous solutions, suspensions, emulsions, or viscous compositions that are buffered to a selected pH. Transdermal and/or topical preparations can be formulated as lotions, gels, sprays, ointments, or other suitable formulations. If nasal or respiratory (mucosal) administration is desired (e.g., aerosol inhalation or insufflation), compositions can be in a form and dispensed by a squeeze spray dispenser, pump dispenser, or aerosol dispenser. Aerosols are usually under pressure by means of a hydrocarbon. Pump dispensers can in some embodiments dispense a metered dose or a dose having a particular particle size, as desired.

When in the form of solutions, suspensions, or gels, vaccine formulations of the compositions of the presently disclosed subject matter can typically contain a major amount of water (in some embodiments, purified water) in addition to the active ingredient(s). Minor amounts of other ingredients such as pH adjusters, emulsifiers, dispersing agents, buffering agents, preservatives, wetting agents, jelling agents, colors, and the like can also be present.

The compositions of the presently disclosed subject matter as vaccines are in some embodiments isotonic with the blood or other body fluid of the recipient. The isotonicity of the compositions can be attained using sodium tartrate, propylene glycol, or other inorganic or organic solutes. In some embodiments, isotonicity is attained using sodium chloride. Buffering agents can be employed, including but not limited to acetic acid and salts, citric acid and salts, boric acid and salts, and phosphoric acid and salts. Parenteral vehicles include but not limited to sodium chloride solution, Ringer's dextrose, dextrose and sodium chloride, lactated Ringer's, and/or fixed oils can also be employed. Intravenous vehicles include fluid and nutrient replenishers, electrolyte replenishers (such as those based on Ringer's dextrose), and the like.

Viscosity of the compositions of the presently disclosed subject matter as vaccines can be maintained at the selected level using a pharmaceutically acceptable thickening agent. Methylcellulose is an exemplary thickening agent because it is readily and economically available and is easy to work with. Other suitable thickening agents include, for example, xanthan gum, carboxymethyl cellulose, hydroxypropyl cellulose, carbomer, and the like. The desired concentration of the thickener can depend upon the agent selected. In some embodiments, an amount of thicken is employed to achieve a pre-selected viscosity. Viscous compositions are normally prepared from solutions by the addition of such thickening agents.

A pharmaceutically acceptable preservative can be employed to increase the shelf life of the compositions. Benzyl alcohol can be suitable, although a variety of preservatives including, but not limited to parabens, thimerosal, chlorobutanol, or benzalkonium chloride can also be employed. A suitable concentration of the preservative can be from 0.02% to 2% based on the total weight although there can be appreciable variation depending upon the agent selected.

### VI. In Vitro Uses

In addition to treatment methods and other in vivo uses, the penetrating peptide compositions disclosed herein can also be used in the context of in vitro experimentation. For example, the penetrating peptides disclosed herein can be used to deliver any of a wide variety of active agents as discussed herein, as well as potential active agents, into viable cells in vitro to determine the potential therapeutic effect, toxicity, etc. of the active agent or potential active agent. For this reason, the penetrating peptides and penetrating peptide compositions of the present disclosure can be useful in the context of drug testing and/or screening.

In some embodiments, penetrating peptide compositions as described herein can be used in in vitro gene silencing experiments, e.g., by introducing a penetrating peptide-interfering RNA conjugate directed to a gene target and monitoring the effect on gene expression.

Additional in vitro uses can include the use of penetrating peptides as disclosed herein conjugated or associated with one or more labeling agents (e.g., fluorescent agents or radioactive labels) or one or more labeling agent carriers in order to label viable cells in vitro.

### Example

The following EXAMPLE is put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

Phage Display Library Screening for Skin Penetrating Peptides (SPPs)

Phage Display Library (PDL) screening was performed on full thickness porcine skin using Franz diffusion cells. Porcine skin was sandwiched in between the donor and receptor compartments in such a way that SC faced the donor compartment. Phosphate-buffered saline (PBS: 10 mM), pH 7.4 (12 ml) was added in the receptor compartment. The skin was equilibrated at 37° C. for 0.5-1 hours before the experiment. The skin integrity was determined by measuring the skin conductivity.

To perform PDL screening, 1×10¹¹ plaque forming units (pfu) of phages from a 12-mer phage display peptide library (PDL) were added into the donor compartment and covered with parafilm to maintain the occlusive condition. Phage particles which permeated across the skin were collected from the receptor chamber after 24 hours and amplified using a standard M13 phage amplification protocol. Amplified pool of phages (1×10¹¹ pfu) was loaded again in the donor chamber for another round of screening. In this way, three rounds of screenings were performed.

After third round of screening, DNAs were isolated from phages, which crossed the skin consistently in higher numbers, and sent for DNA sequencing to analyze the peptide present on their surface. After three round of screening, seven (7) peptide sequences that consistently crossed the skin were identified (see Table 3). Among these, one sequence (HIITDPNMAEYL; SEQ ID NO: 1) was found that crossed the skin at very high frequency. The identified peptides, SEQ ID NOs: 1-7, are referred to herein as skin penetrating peptides (SPPs).

TABLE 3
SPPs Identified After Three Rounds of PDL
Screening on Porcine Skin

| Peptide Sequence | Frequency* |
|---|---|
| HIITDPNMAEYL (SEQ ID NO: 1) | 8 out of 31 |
| SYTQRADSTTLH (SEQ ID NO: 2) | 3 out of 31 |
| GYGFSNTNSFFV (SEQ ID NO: 3) | 3 out of 31 |
| SHMQNRPASDEH (SEQ ID NO: 4) | 3 out of 31 |
| YNAGSILENNF (SEQ ID NO: 5) | 2 out of 31 |
| LVPDRMTAISRA (SEQ ID NO: 6) | 2 out of 31 |
| NSLRNYDFLITM (SEQ ID NO: 7) | 2 out of 31 |

*″Frequency″ refers to how many times a same peptide sequence was identified, when 31 peptides were randomly picked from the receptor compartment of four different skin samples, after third round of screening during (n = 4).

Interestingly, one SPP sequence (HIITDPNMAEYL; SEQ ID NO: 1) has more than 80% similarity to a Rotavirus NSP4 glycoprotein. Rotavirus NSP4 glycoprotein is known to penetrate/alter/destabilize plasma membrane and cause leakage in epithelial cells. Mechanistically, Rotavirus NSP4 glycoprotein binds to extracellular matrix proteins such as fibronectin. It is possible that identified SPP when removed from the phage surface would have a potential to enhance penetration across the skin and cell membranes and possibly act via binding to ECM proteins.

What is claimed is:

1. A composition comprising a peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7), wherein the peptide is associated with and/or conjugated to an active agent and/or a carrier comprising the active agent, and further wherein the composition is capable of penetrating a stratum corneum (SC) layer when contacted therewith or penetrating a cell when contacted therewith.

2. The composition of claim 1, wherein the composition is capable of penetrating the SC layer.

3. The composition of claim 1, wherein the active agent comprises a protein, a nucleic acid, a pharmaceutical compound, a detectable agent, or a nanoparticle.

4. The composition of claim 3, wherein the protein comprises an antibody or a fragment thereof comprising at least one paratope.

5. The composition of claim 3, wherein the active agent comprises a pharmaceutical compound.

6. The composition of claim 3, wherein the active agent comprises a detectable agent.

7. The composition of claim 1, wherein the carrier comprises a nanoparticle.

8. The composition of claim 1, wherein the peptide is conjugated to the active agent and/or the carrier.

9. The composition of claim 1, wherein the peptide is conjugated to the carrier.

10. The composition of claim 1, wherein the peptide is associated with the active agent and/or the carrier via hydrophobic, electrostatic, or van der Walls interactions.

11. The composition of claim 1, wherein the peptide is from 9 to 11 amino acids in length.

12. The composition of claim 1, wherein the peptide is from about 12-15 amino acids in length.

13. The composition of claim 1, wherein the peptide is from about 16-19 amino acids in length.

14. An isolated peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7).

15. The isolated peptide of claim 14, wherein the peptide comprises repeat units of one or more of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7).

16. The isolated peptide of claim 15, wherein the unit is repeated 2 to 50 times.

17. The isolated peptide of claim 16, wherein each unit is separated by an intervening peptide sequence.

18. The isolated peptide of claim 14, wherein the isolated peptide is from 9 to 11 amino acids in length.

19. The isolated peptide of claim 14, wherein the isolated peptide from about 12-15 amino acids in length.

20. The isolated peptide of claim 14, wherein the isolated peptide is from about 16-19 amino acids in length.

21. A method of delivering an active agent to a subject, comprising: administering to the subject a composition comprising a peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7), wherein the peptide is conjugated to and/or associated with an active agent or a carrier comprising the active agent, and wherein the composition is capable of penetrating the stratum corneum (SC) of the subject and/or penetrating a cell of the subject.

22. The method of claim 21, wherein the composition is capable of penetrating the SC of the subject.

23. The method of claim 21, wherein the administration is topical administration.

24. The method of claim 21, wherein the composition is capable of penetrating the cellular membrane of a cell selected from the group consisting of a viable non-human animal cell, a viable human cell, a viable epidermal cell, a viable dermal cell, and a viable immunological cell.

25. The method of claim 21, wherein the active agent comprises a protein, a nucleic acid, a pharmaceutical compound, a detectable agent, and/or a nanoparticle.

26. The method of claim 25, wherein the protein comprises an antibody or a fragment thereof comprising at least one paratope.

27. The method of claim 25, wherein the active agent comprises a pharmaceutical compound.

28. The method of claim 35, wherein the active agent comprises a detectable agent.

29. The method of claim 21, wherein the carrier comprises a nanoparticle.

30. The method of claim 21, wherein the peptide is conjugated to the active agent.

31. The method of claim 21, wherein the peptide is conjugated to the carrier comprising the active agent.

32. The method of claim 21, wherein the peptide is associated with the active agent or the active agent carrier comprising the active agent, via hydrophobic, electrostatic and/or van der Walls interactions.

33. A method of treating a subject having a disease or disorder, comprising: administering to the subject a composition comprising a peptide comprising, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7), wherein the peptide is conjugated to and/or associated with a dermatological active agent and/or a dermatological active agent carrier comprising the active agent, and further wherein the composition is capable of penetrating the stratum corneum (SC) of the subject or penetrating a cell of the subject.

34. The method of claim 33, wherein the composition is capable of penetrating the SC of the subject and penetrating the cell of the subject.

35. The method of claim 33, wherein the administration is topical administration.

36. The method of claim 33, wherein the composition is capable of penetrating the cellular membrane of a cell.

37. The method of claim 33, wherein the active agent comprises a protein, a nucleic acid, a pharmaceutical compound, a detectable agent, and/or a nanoparticle.

38. The method of claim 37, wherein the active agent comprises a pharmaceutical compound.

39. The method of claim 37, wherein the active agent comprises a detectable agent.

40. The method of claim 33, wherein the carrier comprises a nanoparticle.

41. The method of claim 33, wherein the peptide is conjugated to the active agent.

42. The method of claim 33, wherein the peptide is conjugated to an active agent carrier comprising the active agent.

43. The method of claim 33, wherein the peptide is associated with the active agent or the active agent carrier comprising the active agent, via hydrophobic, electrostatic or van der Walls interactions.

44. A composition comprising a peptide consisting essentially of or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7), wherein the peptide is associated with and/or conjugated to an active agent or a carrier comprising the active agent, and further wherein the composition is capable of penetrating a stratum corneum (SC) layer when contacted therewith and/or penetrating a cell when contacted therewith.

45. A method for inducing an immune response in a subject, the method comprising administering to the subject a composition comprising a peptide conjugated to and/or associated with an antigen to which an immune response in the subject is desired and/or a carrier comprising the antigen, wherein the peptide comprises, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7), and further wherein the composition is capable of penetrating the stratum corneum (SC) of the subject and/or penetrating a cell of the subject to deliver the antigen across the SC or into the cell.

46. The method of claim 45, wherein the composition is capable of penetrating the SC of the subject.

47. A composition comprising a peptide conjugated to and/or associated with an antigen to which an immune response in the subject is desired and/or a carrier comprising the antigen, wherein:
(i) the peptide comprises, consisting essentially of, or consisting of an amino acid sequence selected from the group consisting of HIITDPNMAEYL (SEQ ID NO: 1), SYTQRADSTTLH (SEQ ID NO: 2), GYGFSNTNSFFV (SEQ ID NO: 3), SHMQNRPASDEH (SEQ ID NO: 4), AYNAGSILENNF (SEQ ID NO: 5), LVPDRMTAISRA (SEQ ID NO: 6), and NSLRNYDFLITM (SEQ ID NO: 7);
(ii) the antigen is present in the composition in an amount sufficient to elicit an immune response in a subject to the antigen; and
(iii) the composition penetrates the stratum corneum (SC) of the subject and/or a cell of the subject to deliver the antigen to the subject's immune system.

48. The composition of claim 47, wherein the composition is in a stable, dry particulate form comprising the peptide and the antigen and/or the carrier.

49. The composition of claim 47, wherein the composition further comprises an adjuvant; a stabilizer, optionally a stabilizer selected from the group consisting of a protein stabilizer, a sugar, and a sugar derivative; a pharmaceutically acceptable carrier or diluent; or any combination thereof.
